(19) United States
(12) Patent Application Publication
(10) Pub. No.: US 2009/0118589 A1
(43) Pub. Date: May 7, 2009

(54) HEALTH MANAGEMENT SUPPORT SYSTEM AND RECORDING MEDIUM

(76) Inventors: Hiromu Ueshima, Shiga (JP); Yoshihiro Iljima, Shiga (JP)

Correspondence Address: JEROME D. JACKSON (JACKSON PATENT LAW OFFICE), 211 N. UNION STREET, SUITE 100, ALEXANDRIA, VA, 22314, US

(21) Appl. No.: 11/722,725

(22) PCT Filed: Dec. 27, 2005
(86) PCT No.: PCT/JP2005/023949

§ 371 Date: Jun. 3, 2008

Related U.S. Application Data
(60) Provisional application No. 60/639,670, filed on Dec. 28, 2004.

(51) Int. Cl. A61B 5/00

(52) U.S. Cl. 600/300

(57) ABSTRACT

A health management support system capable of showing the change in biometric information in a form which can be easily understood is provided. The health management support system includes: a storage unit for storing a measurement value of biometric information such as a body weight in association with the measurement date; a symbol assignment processing unit for calculating a change tendency of measurement values of the biometric information stored over a plurality of periods each of which consists of previous days including a predetermined measurement day as the last day of the each period and assigning one of arrow symbols 360, 362, 364 and 366, which are prepared in advance, to the plurality of periods in accordance with the change tendency as calculated; and a display processing unit for displaying the symbols 360, 362, 364 and 366 assigned to the plurality of periods by the symbol assignment unit on the same display screen 340.

### TECHNICAL FIELD

The present invention relates to a system for providing assistance to maintain good health and, more particularly, to a health management support system for providing assistance to maintaining good health by accumulating and analyzing biometric information such as body weight, which is measured in relation to health, and displaying the result of the analysis on a monitor or the like.

### BACKGROUND ART

As people become more health conscious in recent years, a variety of health care devices have been developed. Generally, such health care devices fall into three types. The first type is a device serving to acquire the information on body condition for use in maintaining good health, the second type is a device used to perform exercise effective for maintaining good health, and the third type is a device used to perform a treatment for alleviating some sort of symptom.

Typical examples of the first type devices include body weight scales, measurement devices for measuring body fat percentages, blood pressure manometers, measurement sensors for measuring blood glucose, and the like. Typical examples of the second type devices include indoor running machines, indoor walking machines, game machines with exercise and the like for use in performing indoor exercise associated with some physical loads. Typical examples of the third type devices include massage chairs, foot baths, shoulder tapping devices and a number of other similar devices.

However, for maintaining good health, it is most fundamental to know the condition of own body. Accordingly, the first type of health care devices is believed to be most fundamental.

However, in the case of such a health care device, it is an outstanding issue how to design a presentation method of giving the user the information acquired by the device. In particular, this kind of information is often simple numeral information such as a body weight, a body fat percentage or the like, and thereby it is important how to make the user understand the meanings of the information.

Specifically, while the long term variation of body weight or the like may be problematic, the body weight or the like can vary within a day. It is therefore not so easy to show the information in order that the meanings of the information can be easily understood. Because of this, various body weight scales designed to facilitate understanding of the significance of body weight changes have been proposed.

Patent Document 1 discloses a body weight scale which records body weights together with the time slots in which the measurement is performed. By this configuration, it is possible to display a graph in which body weights are plotted in relation to the time slots. This display makes it possible to know the change tendency of body weight within a day. Also, the long-term change in body weight can be known by displaying this graph to cover a certain period of time. Since the body weight change can be visually shown, it is said that the general management of dietary habit can be easily performed by visually and intuitively understanding information other than the number of calories taken in, for example, missing a meal and the time of eating a meal, with reference to the profile of the graph.

Patent Document 2 discloses a body weight scale capable of showing the daily, weekly and monthly changes in body fat percentage and body weight in a graph at the same time.

[Patent Document 1] Japanese Patent Published Application No. Hei 9-21689 (FIG. 5, paragraphs 0093 to 0100)

[Patent Document 2] Japanese Patent Published Application No. 2001-190514

### DISCLOSURE OF INVENTION

### Problems to be Solved by the Invention

However, in the case of the body weight scales disclosed in Patent Document 1 and Patent Document 2, there is a problem in that it cannot clearly be grasped what is meant by the change in body weight unless the significance of the profile of the graph can be understood. Also, if the graphical representation is provided for a plurality of periods in contradistinction, a substantially large area of a display screen is occupied. However, such a large area may not always be available. Accordingly, a system for managing the body weight is needed which can provide a display screen in a form which facilitates the understanding in a more easily and intuitively manner.

Furthermore, while there is a conventional body weight scale capable of displaying the result of measurement, calculating the amount of energy to be consumed, and displaying the result of the calculation, there is no conventional body weight scale capable of making the user aware of, for example, what could have caused him to promote body weight gain.

Such a problem does not always relate to the body weight. As has been discussed above, similar problems can occur also in relation to the biometric information about the body fat percentage, the blood glucose level, the blood pressure, or any other health factor. Accordingly, there is a need to provide a health management support system capable of showing the change in biometric information in a form which can be easily understood, with respect to the previous biometric information, a health management support system enabling the user to readily use the biometric information as obtained, and a health management support system capable of making the user aware of what could have caused the change in the previous biometric information.

It is therefore an object of the present invention to provide a health management support system capable of showing the change in biometric information as measured in a form which is easier to understood than ever before.

It is another object of the present invention to provide a health management support system capable of showing the change in biometric information as measured in a form which is easier to understood and use the change than ever before.

It is a further object of the present invention to provide a health management support system capable of showing the change in biometric information as measured in a form which is easier to understood and use the change than ever before, and making the user aware of what could have caused the change.

### Means to Solve the Problems

In accordance with a first aspect of the present invention, a system for supporting health management comprises: a storing unit operable to store a measurement value of predetermined biometric information in association with the measurement date thereof; a symbol assignment unit operable to calculate the change tendencies of the measurement values of the biometric information stored in the storing unit over a plurality of periods each of which consists of previous days including a predetermined measurement day as the last day of the each period, and assign one symbol of a plurality of types of symbols, which are prepared in advance, to each of the plurality of periods in accordance with the change tendency as calculated; and a display unit operable to display the symbols assigned to the plurality of periods by the symbol assignment unit respectively in the same display screen.

The measurement value of the predetermined biometric information is stored in the storage unit in association with the measurement date thereof. The symbol assignment unit assigns one symbol of the plurality of types of symbols to each of the plurality of periods in accordance with the change tendencies of the measurement values of the biometric information stored in the storing unit over the plurality of periods each of which consists of previous days including the predetermined measurement day as the last day of the each period. The display unit displays the symbols assigned to the plurality of periods in the same screen. The change tendencies over the plurality of periods are shown by the symbols and displayed in the same screen. The change tendency of each period is displayed in the form of a symbol for easier understanding. In addition to this, the change tendencies over the plurality of periods are contrasted in the same screen. Because of this, the change tendency and course of the biometric information can be easily and readily understood.

Preferably, the symbols include arrow symbols having inclinations which reflect the change tendencies of the measurement values of the biometric information respectively.

In many cases, the measurement values of the biometric information are represented by numerical numbers, and the variations in the numerical numbers have certain meanings for the health care management. The symbol in the form of an arrow serves to appropriately represent the change tendency of such numerical numbers, and thereby the change tendency of the measurement value of the biometric information becomes more easy to understand.

More preferably, the respective symbols are given different colors respectively in accordance with the calculated change tendencies.

Color has a significant meaning when a person recognizes things. In addition, color can be discriminated even if a shape cannot be confirmed. Because of this, when a plurality of symbols indicative of the change tendencies of the biometric information is displayed in a screen, it is possible to readily understand the meanings of the symbols.

Furthermore, preferably, the predetermined biometric information is all or part of body weight, body fat percentage, blood pressure, blood glucose level, dimensions of predetermined areas of body (for example, waist, hip, bust, thigh, body height and/or the like), number of pulses, body temperature, number of steps, consumed calories and taken calories. The biometric information may also include information which is based on a function of all or part of the above (for example, BMI (Body Mass Index) and so forth).

For example, the gains and losses in body weight are very important information for the health care management. By displaying the above items of the information in the same screen in any way as described above, it is possible to easily know, through a plurality of periods, the tendency of the gains and losses in body weight, and the change in this tendency. The same discussion applies to other biometric information.

Preferably, the symbol assignment unit comprises: a change tendency calculating unit operable to calculate the change tendencies of the measurement values of the biometric information stored in the storing unit over a plurality of periods each of which consists of days including a predetermined measurement day as the last day of the each period; and an assignment unit operable to assign one symbol of a plurality of types of symbols, which are prepared in advance, to each of the plurality of periods in accordance with the change tendency calculated by the change tendency calculating unit, and wherein the change tendency calculating unit comprises: a first moving average calculating unit operable to calculate the moving average of the biometric information, for each of the plurality of periods, over a period whose length is same as that of the each of the plurality of periods and whose last day is one day before the first day of the each of the plurality of periods; and a calculating unit operable to calculate, for each of the plurality of periods, the difference between the moving averages calculated by the second moving average calculating unit and the first moving average calculating unit.

When the change tendency of the biometric information is calculated, the biometric information of the predetermined measurement date is contrasted with the biometric information of the first day of the each period in terms of the moving averages thereof. The variation in each measurement substantially affects the result if each measurement value is used alone. However, by the use of moving averages, the influence of such variation can be minimized to make it possible to more appropriately know the change tendency. Furthermore, in both the measurement date and the date to be contrasted, the moving average is calculated by the use of the measurement values over the period having the same length as the each period. The change tendency can be accurately evaluated by calculating the moving averages in the same condition in this manner.

The assignment unit may include a unit operable to assign, for each of the plurality of periods, an appropriate one of the plurality of types of symbols to the each of the plurality of periods in accordance with which of a plurality of predetermined ranges includes the difference between the moving averages of the each of the plurality of periods calculated by the unit operable to calculate the difference.

In this case, the types of symbols are provided in the same number as there are the plurality of ranges. It can therefore be avoided that the number of the types of symbols unnecessarily increases to hinder the understanding of the change tendency.

More preferably, the plurality of ranges is determined by a function of the biometric information stored in the storage unit and the length of the period to be considered.

For example, the body weight is very important information for the health care management. Also, there is the possibility that the change tendency varies depending upon the body weight and the period to be considered. Thereby, it is considered that the change tendency of body weight can be more appropriately indicated by changing the range of the change in body weight for use in assigning symbols in accordance with the body weight and the period to be considered. The same discussion applies to other biometric information.

In accordance with a second aspect of the present invention, a system for supporting health management comprises: a storing unit operable to store information used to identify the difference between a measurement value of predetermined biometric information in a first time slot and a measurement value of the predetermined biometric information in a second time slot respectively in association with the measurement date thereof; a first graph displaying unit operable to display a graph in a first graph form showing the change in the measurement value in the first time slot stored in the storage unit for a predetermined period; and a second graph displaying unit operable to graph the difference between the measurement value in the first time slot and a measurement value of the predetermined biometric information in a second time slot stored in the storage unit within the predetermined period in a second graph form, and display the graph of the difference to be superimposed over the graph displayed by the first graph displaying unit, wherein the second graph displaying unit displays a graph, using the position of the measurement value in the first time slot for each measurement date as a base point, to show the difference between the measured value in the second time slot and the measured value in the first time slot in the each measurement date.

The storage unit daily stores the measurement value of the predetermined biometric information in a first time slot and the information used to identify the difference between the measurement value in the first time slot and the measurement value of the predetermined biometric information in the second time slot respectively in association with the measurement date thereof. The first graph displaying unit displays the graph in the first graph form showing the change in the measurement value in the first time slot for the predetermined period. The second graph displaying unit displays the graph in the second graph form showing the difference between the measurement value in the first time slot and the measurement value in the second time slot. This graph is displayed to be superimposed over the graph displayed by the first graph displaying unit. In doing so, the second graph displaying unit displays the graph to show the difference by using the position of the measurement value in the first time slot for each measurement date as a base point. It is possible to know the change tendency of the measurement value in the first time slot with reference to the graph provided by the first graph displaying unit on the basis of the measurement value in the first time slot of the each measurement date. Furthermore, the difference between the measurement value in the first time slot and the measurement value in the second time slot is displayed in the second graph form by the second graph displaying unit with the point of the graph provided by the first graph displaying unit corresponding to the measurement date as a base point. Accordingly, the variation in the each measurement date can be visualized and expressed to make easy to understand.

Preferably, the second graph displaying unit comprises: a determination unit operable to determine whether or not the difference in each measurement date satisfies a predetermined condition; and an overlapping displaying unit operable to graph the difference in the each measurement date within the predetermined period in the second graph form, and display the graph of the difference to be superimposed over the graph displayed by the first graph displaying unit, wherein the overlapping displaying unit displays the graph using the position of the measurement value in the first time slot for each measurement date as a base point and using a different color in accordance with the result of determination by the determination unit.

The second graph displaying unit displays the graph using a different color for the each measurement date in accordance with whether or not a predetermined condition is satisfied by the difference between the measurement value in the first time slot and the measurement value in the second time slot. In the case where the difference between the measurement value in the first time slot and the measurement value in the second time slot has a substantial meaning for the health care management, by using such graph representation, it is possible to intuitively grasp the significance of the change in the measurement value in each measurement date and perform more effective health care management.

More preferably, the predetermined biometric information is all or part of body weight, body fat percentage, blood pressure, blood glucose level, dimensions of predetermined areas of body (for example, waist, hip, bust, thigh, body height and/or the like), number of pulses, body temperature, number of steps, consumed calories and taken calories; the first time slot is selected as a time slot which is earlier than the second time slot in each measurement date; and the determination unit includes a unit operable to determine, for each measurement date, whether or not the difference of the each measurement date is greater than a predetermined threshold value. The biometric information may also include information which is based on a function of all or part of the above (for example, BMI and so forth).

For example, it has a substantial meaning whether or not the difference between the measurement value in the first time slot (for example, morning) and the measurement value in the second time slot (for example, night) is greater than the threshold value. Accordingly, by changing the color of the second graph depending upon whether or not this condition is satisfied, it is possible to visually and easily discriminate whether or not the differential body weight is greater than the threshold for each measurement date and easily perform the health care management. The same discussion applies to other biometric information.

Furthermore, preferably, the displaying unit uses a color of red for graphing the difference in response to the determination that the difference of the each measurement date is greater than the predetermined threshold value.

This system may include a unit operable to display a particular screen (comment screen), which is prepared in advance for managing body weight, in response to the determination that the difference of a certain measurement date is greater than the predetermined threshold value.

Preferably, this system further includes a unit operable to calculate the predetermined threshold value as a function of the biometric information of a user stored in the storage unit.

More preferably, the displaying unit uses a color of red for graphing the difference in response to the determination that the difference of the each measurement date is greater than the predetermined threshold value.

Still further, preferably, the first graph form is a line graph connecting daily measurement values. The second graph form may be a vertical bar graph consisting of bars each of which has a vertical length in proportion to a daily difference.

In accordance with a third aspect of the present invention, a recording medium is provided for storing a computer program for causing, when executed by a computer connected to a display device, the computer to function as a system for supporting health management, the system comprising: a storing unit operable to store measurement values of predetermined biometric information measured at predetermined intervals in association with the measurement date thereof; a symbol assignment unit operable to calculate the change tendencies of the measurement values of the biometric information stored in the storing unit over a plurality of periods each of which consists of previous days including a predetermined measurement day as the last day of the each period, and assign one symbol of a plurality of types of symbols, which are prepared in advance, to each of the plurality of periods in accordance with the change tendency as calculated; and a display signal generation unit operable to generate a signal for displaying the symbols assigned to the plurality of periods by the symbol assignment unit respectively in the same display screen of the display device.

When the computer program stored in this recording medium is executed by the computer, the system for supporting health management in accordance with the first aspect is realized so that there are the similar advantages.

In accordance with a fourth aspect of the present invention, a recording medium is provided for storing a computer program for causing, when executed by a computer connectable to a display device, the computer to function as a system for supporting health management, the system comprising: a storing unit operable to daily store information used to identify the difference between a measurement value of predetermined biometric information in a first time slot and a measurement value of the predetermined biometric information in a second time slot respectively in association with the measurement dates thereof; a first graph displaying unit operable to display a graph in a first graph form showing the change in the measurement value in the first time slot stored in the storage unit for a predetermined period; and a second graph displaying unit operable to graph the difference between the measurement value in the first time slot and a measurement value of the predetermined biometric information in a second time slot stored in the storage unit within the predetermined period in a second graph form, and display the graph of the difference to be superimposed over the graph displayed by the first graph displaying unit, wherein the second graph displaying unit displays a graph, using the position of the measurement value in the first time slot for each measurement date as a base point, to show the difference between the measured value in the second time slot and the measured value in the first time slot in the each measurement date.

When the computer program stored in this recording medium is executed by the computer, the system for supporting health management in accordance with the second aspect is realized so that there are the similar advantages.

### BRIEF DESCRIPTION OF DRAWINGS

FIG. 1 A view for showing the overall hardware configuration of a health management support system in accordance with an embodiment of the present invention.

FIG. 2 A perspective view showing an adapter 1 and a cartridge 3 of FIG. 1.

FIG. 3 A perspective view showing the adapter 1 as seen from the back side.

FIG. 4 A block diagram showing the internal configurations of the cartridge 3 and the body weight scale 300 of FIG. 1.

FIG. 5 A hardware block diagram showing the adapter 1 of FIG. 1.

FIG. 6 A hardware block diagram showing the cartridge 3 of FIG. 1.

FIG. 7 A flow chart showing the process of measuring a body weight by the MCU 320 of the body weight scale 300 of FIG. 4.

FIG. 8 A view showing a communication protocol between the cartridge 3 and the body weight scale 300 of FIG. 4.

FIG. 9 A view schematically showing a body weight tendency display screen displayed on the television monitor 5 by the adapter 1.

FIG. 10 A view schematically showing a chart screen displayed on the television monitor 5 by the adapter 1.

FIG. 11 A view schematically showing a checklist screen displayed on the television monitor 5 by the adapter 1.

FIG. 12 A view schematically showing a radar chart screen as a result of the health check process displayed on the television monitor 5 by the adapter 1.

FIG. 13 A view schematically showing a progress/achievement display screen 480 displayed after daily body weight measurement.

FIG. 14 A flowchart showing the overall process of the health management support system shown in FIG. 1.

FIG. 15 A flowchart showing the first half of the flow chart of the main process performed in step S106 of FIG. 14.

FIG. 16 A flowchart showing the latter half of the flow chart of the main process.

FIG. 17 A flow chart for showing the process of displaying a graph screen.

FIG. 18 A flow chart for showing a health check process.

FIG. 19 A flow chart for showing the process of displaying course and achievement.

FIG. 20 A flowchart showing a flow chart of the process of displaying the body weight tendency.

FIG. 21 A flowchart showing a flow chart of the process of displaying the body weight tendency.

FIG. 22 A flowchart showing a flow chart of the process of displaying the body weight tendency.

FIG. 23 A flowchart showing a flow chart of the process of displaying the body weight tendency.

### EXPLANATION OF REFERENCE NUMERALS

1 . . . adapter, 3 . . . cartridge, 5 . . . television monitor, 35 . . . enter key, 37 . . . arrow keys, 39 . . . cancel key, 45 . . . power switch, 43 . . . reset switch, 91 . . . high speed processor, 93 . . . memory, 300 . . . body weight scale, 302 . . . display unit, 305 . . . remote controller, 306 . . . ROM, 308 . . . serial flash ROM, 310, 312 . . . RF module, 314 . . . EEPROM, 316 . . . real time clock unit, 318 . . . body weight/fat measuring unit, 319 . . . switch unit, 320 . . . MCU

### BEST MODE FOR CARRYING OUT THE INVENTION

A health management support system in accordance with an embodiment of the present invention will be explained in conjunction with the accompanying drawings. Meanwhile, like references indicate the same elements throughout the respective drawing. These same elements have the same names and functions. Therefore, redundant explanation is not repeated for these elements. In the following explanation, the body weight is taken as an example of the information with respect to which the health management is performed. However, the present invention can be applied not only to the body weight but also to any biometric information relating to the health management.

U.S. patent application Ser. No. 10/947,064 and U.S. Pat. No. 6,607,436 both filed by the present applicant are incorporated in this disclosure by reference.

[Hardware Configuration of Health Management System]

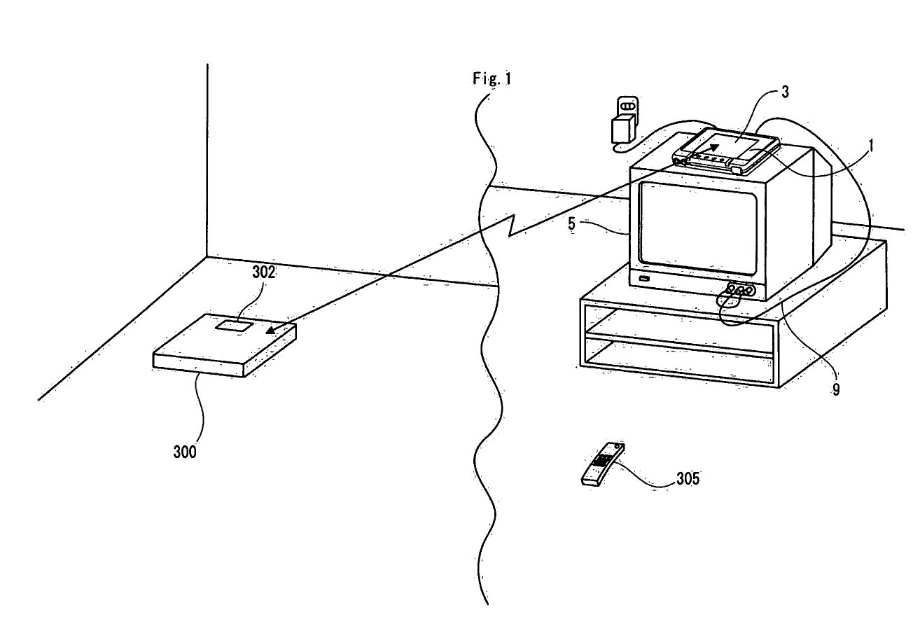

FIG. 1 is a view for showing the overall hardware configuration of the health management support system for use with respect to the body weight in accordance with an embodiment of the present invention. As shown in FIG. 1, this health management support system includes a body weight scale 300 provided with a short-range wireless communication capability, a television monitor 5, a cartridge 3 having a predetermined configuration, an adapter 1 which can be connected to the television monitor 5 and is designed so that the cartridge 3 can be inserted thereto. The cartridge 3 has a wireless communication capability and contains a memory for accumulating the information acquired from the body weight scale 300 and storing a computer program to perform the main processing of the health management support system by outputting images and sounds to the television monitor 5 through the adapter 1, and a high speed processor capable of performing this program.

The upper surface of the body weight scale 300 is provided with a display unit 302 which is used to display a body weight, a body fat percentage, a user number to be described below, and the like. The body weight scale 300 is placed, for example, in a bathroom.

The adapter 1 is placed, for example, on the upper surface of the television monitor 5. The adapter 1 and the television monitor 5 are connected by an AV cable 9. The adapter 1 is provided with an infrared communication port and can be controlled by an infrared remote control 305. A mouse can be used in place of the infrared remote control 305. The television monitor 5 and the adapter 1 with the cartridge 3 inserted therein are placed, for example, in a living room.

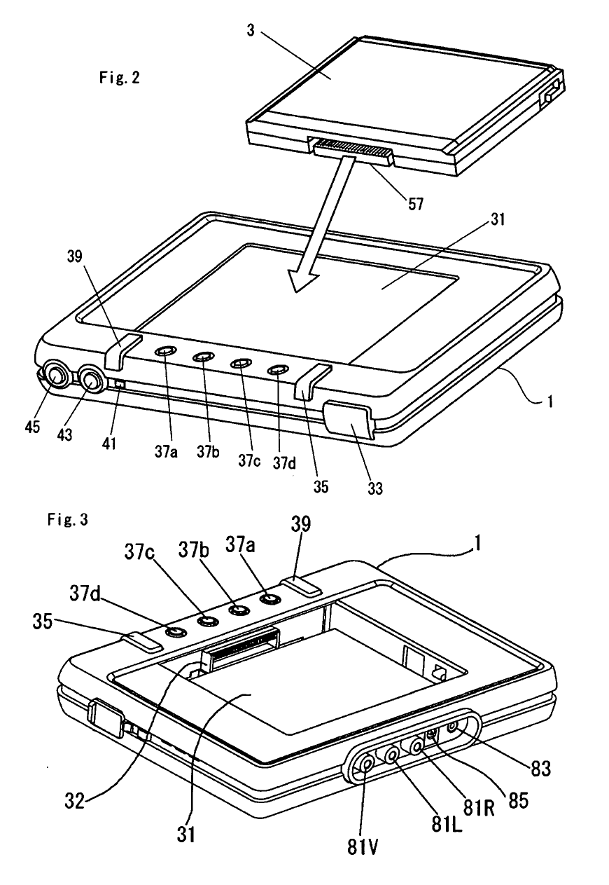

FIG. 2 is a perspective view showing the adapter 1 and the cartridge 3 of FIG. 1. FIG. 3 is a perspective view showing the adapter 1 as seen from the back side.

As shown in FIG. 2, the adapter 1 has a flat rectangular parallelepiped shape with an upper face, a lower face, a right and a left side face, a front and a back face. The adapter 1 is provided with a power supply switch 45, a reset switch 43 and a power lamp 41 in the left hand side of the front face, and an infrared filter 33 in the right hand side of the front face. This infrared filter 33 is a filter capable of removing incident light except infrared light in order to only pass infrared light, and an infrared sensor (not shown in the figure) is located behind this infrared filter 33. On the upper surface of the adapter 1 in the vicinity of the front edge thereof, arrow keys 37a to 37d (a up key, a down key, a right key and a left key) are provided and can be used by the user to move the cursor displayed on the television monitor 5. Also, a cancel key 39 is provided on the left side of the arrow key 37a, and an enter key 35 is provided on the right side of the arrow key 37d. There are a variety of ways to use these arrow keys, cancel key and enter key, however, in the case of the present embodiment, these keys are used to select a desired process to be described below.

As shown in FIG. 3, an AV jack 83, a power jack 85, a video jack 81V, an L channel audio jack 81L and an R channel audio jack 81R are provided in the back face of the adapter 1. Incidentally, the term “AV jack 81” is used to generally represent the video jack 81V, the L channel audio jack 81L and the R channel audio jack 81R. The AV jack 83 is an external output terminal, and connected to an external input terminal of the television monitor 5 through the AV cable 9. On the other hand, the AV jack 81 is an input terminal which can be connected to the output terminal of a variety of external equipments (for example, DVD (digital versatile disc) player).

An opening is formed in the center position of the upper surface of the adapter 1. A top plate 31 is disposed in the opening so that its upper face is approximately flush with the upper face of the adapter 1. Inside the adapter 1, there is an elevator mechanism (not shown in the figure) which supports and urges upward the top plate 31 so that the upper face of the top plate 31 is located at the height as described above when no force is exerted upon the upper face of the top plate 31. The top plate 31 is supported to move up and down in the opening by this elevator mechanism. Namely, when a force is exerted upon the top plate 31 from the above, the top plate 31 is moved downward in the opening, while being supported by the elevator mechanism, and stopped in the predetermined position by the action of the elevator mechanism. When the force is removed, the top plate 31 moves upward in the opening, while being supported, and stops in the predetermined position where its upper face is approximately flush with the upper face of the adapter 1.

As shown in FIG. 3, a connector 32 is provided in the opening of the adapter 1 at a location near the front side of the adapter 1. On the other hand, the cartridge 3 is provided with a connector section 57 on the front side of the cartridge 3. The cartridge 3 can be installed in the adapter 1 by placing and pushing down the cartridge 3 on the top plate 31, and sliding the cartridge 3 toward the front face of the cartridge 3 (refer to FIG. 1) in order to connect the connector section 57 of the cartridge 3 to this connector 32.

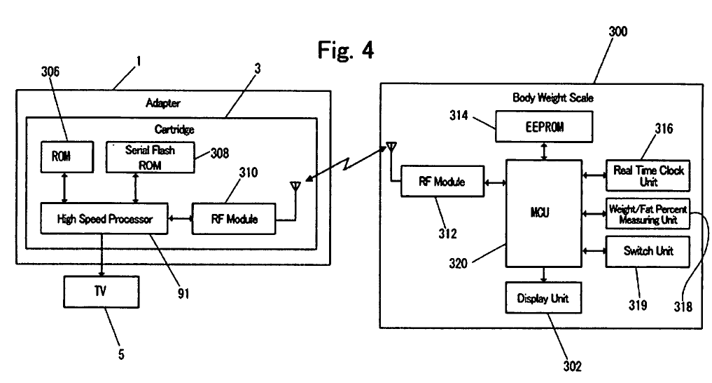

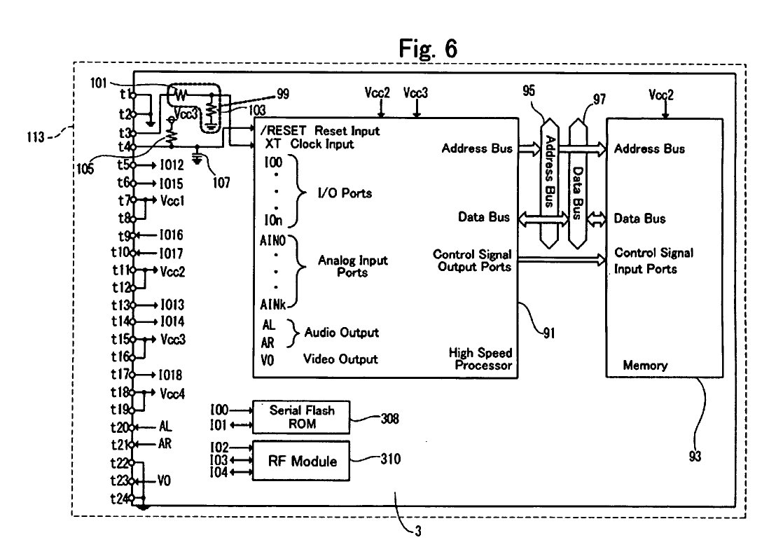

FIG. 4 is a block diagram showing the internal configurations of the cartridge 3 and the body weight scale 300 of FIG. 1. As shown in FIG. 4, the cartridge 3 includes a ROM 306, a serial flash ROM (read only memory) 308, a high speed processor 91 and an RF (radio frequency) module 310. The details of the cartridge 3 will be described with reference to FIG. 6.

The RF module 310 is connected to I/O ports of the high speed processor 91 including an I/O port assigned for transmitting and receiving data, an I/O port assigned for performing request and response operations between the high speed processors 91 and the RF module 310, and an I/O port assigned for supplying a clock signal from the high speed processor 91. Then, the high speed processor 91 and the RF module 310 exchange data in accordance with a communication protocol employed therebetween.

The ROM 306 stores a program, image data, sound data and so on, and the high speed processor 91 performs a variety of arithmetic operations in accordance with this program and generates a video signal VD on the basis of the image data and audio signals “AL1” and “AR1” on the basis of the sound data. The serial flash ROM 308 stores user data which is transmitted from the body weight scale 300, input through the infrared remote control 305 and the adapter 1, and so on.

In the case of the present embodiment, the serial flash ROM 308 stores the data of each user, which includes personal information (name comprising six letters, gender, date of birth, password comprising four letters, system of unit as selected, and the like), body height, basal body weight, body weight daily measured at morning and at night, body fat percentage, intensity of daily activity, number of steps, number of calories as taken in, number of calories as consumed, so-called three sizes (measurements), blood pressure (high/low), number of heart beat, basal body temperature, and the results of clearing pop-up quiz to be described below. Of these, the personal information, the body height, the intensity of daily activity, the basal body weight and so on are input by the user during registration. With respect to the body weight, the data transmitted from the body weight scale 300 is recorded for a decade. With respect to the body fat percentage, the number of steps, the number of calories, the three sizes, the blood pressure and the basal body temperature, the data daily input is recorded together with the body weight data for a decade in accordance with the user operation. Also, with respect to the results of clearing pop-up quiz, the data at the beginning of a month is recorded for up to a maximum of 12 months.

Meanwhile, in the case of the present embodiment, it is assumed that only the body weight is measured to obtain data which is used, and other daily measured data is manually input. Of course, the present invention is not limited to this specific embodiment. If an appropriate measurement instrument is provided, the other measured data changing from day to day can be automatically accumulated by transmitting the data measured by the measurement instrument to the cartridge 3 in the same manner as the body weight measured by the body weight scale 300.

Incidentally, of the information as accumulated here, the body weight data is recorded separately as the data measured in a first time slot, i.e., morning, and a second time slot, i.e., night. The first time slot and second time slot are not clearly defined, but have to be determined at least in order that the first time slot is a time slot earlier than the second time slot. If possible, it is preferred to determine the first time slot as a time slot just after the wake-up time, and the second time slot just before the bedtime. In this case, even if measured after midnight, the data is recorded as the body weight change of the day.

On the other hand, the body weight scale 300 includes a MCU 320, an RF module 312, an EEPROM 314, a display unit 302, a real time clock unit 316, a switch unit 319 and a body weight/fat measuring unit 318. The MCU 320 performs a variety of arithmetic operations in accordance with a program which is stored in a ROM incorporated therein, and controls the respective function blocks inside of the body weight scale 300 respectively.

The body weight/fat measuring unit 318 measures the body weight and body fat percentage of the user who gets on the body weight scale 300, converts measured data into digital data, and outputs the digital data to the MCU 320. On the basis of the body weight and body fat percentage as input, the MCU 320 displays the numerical values of the body weight and body fat percentage on the display unit 302. The real time clock unit 316 measures the current date and the current time, and outputs the information to the MCU 320. The MCU 320 stores the dates and times in the EEPROM 314 separately for the respective users in association with the body weight and body fat percentage data as input from the body weight/fat measuring unit 318.

The MCU 320 and the RF module 312 exchange data in accordance with a communication protocol employed therebetween. In the case of the present embodiment, the switch unit 319 includes five switches to which number 1 to number 5 are assigned as user numbers, and a detection switch which detects whether or not the user is getting on the body weight scale 300. When the switch corresponding to one of the user numbers 1 through 4 is operated, if a user is registered in association with this number, the body weight measurement process for the user is performed. When operating the switch corresponding to the user number 5 or one of the user numbers to which no user is registered, or when a user directly gets on the body weight scale 300 without operation of any switch, the user is treated as a guest, and the body weight and body fat percentage are simply measured. Namely, the health management process is not performed for a guest.

The RF module 310 of the cartridge 3 and the RF module 312 of the body weight scale 300 exchange data through radio waves in accordance with a communication protocol employed therebetween. The health management support program stored in the cartridge 3 accumulates the data transmitted from the body weight scale 300 in this manner, and performs the health management support process also with reference to the data input from the user.

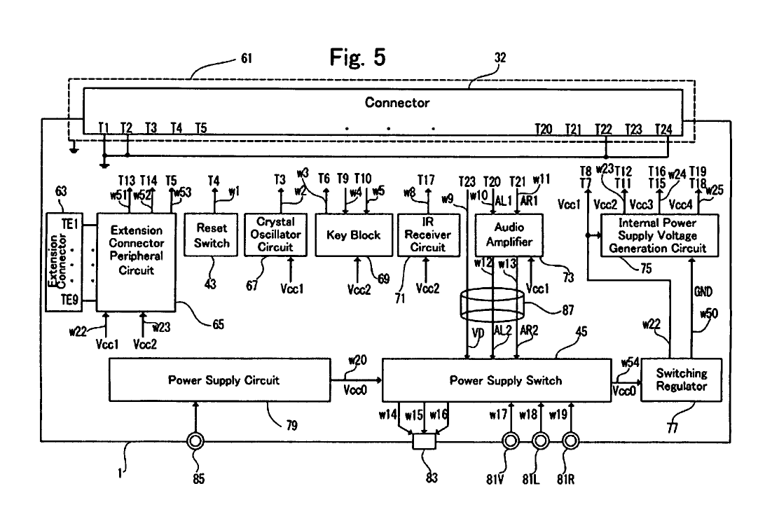

FIG. 5 is a block diagram showing the internal configuration of the adapter 1. As shown in FIG. 5, the adapter 1 includes the connector 32 as described above, and further includes an extension connector 63, an extension connector peripheral circuit 65, the reset switch 43, a crystal oscillator circuit 67, a key block 69, an infrared signal receiver circuit (IR receiver circuit) 71, an audio amplifier 73, an internal power supply voltage generation circuit 75, a power supply circuit 79 comprising an AC/DC converter and the like, the power supply switch 45, a switching regulator 77, the power jack 85, the AV jack 83, the video jack 81V, the L channel audio jack 81L, and the R channel audio jack 81R.

The connector 32 has 24 terminals T1 to T24 and is covered by a shield member 61 which is grounded. The terminals T1, T2, T22 and T24 of the connector 32 are grounded.

The AC voltage as supplied from a power cable (not shown in the figure) is supplied to the power supply circuit 79 through the power jack 85. The power supply circuit 79 converts the AC voltage as given to a DC voltage, which is then output to a line w20 as a power supply voltage Vcc0.

When turned on, the power supply switch 45 connects the line w20 and a line w54 to give the switching regulator 77 the power supply voltage Vcc0, and gives the AV jack 83 a video signal “VD” from a line w9 and audio signals “AL2” and “AR2” from the lines w12 and w13 respectively through lines w14, w15 and w16. Accordingly, the video signal “VD” and the audio signals “AL2” and “AR2” are given to the television monitor 5 through the AV cable 9, while the television monitor 5 displays a picture with sounds output from a speaker (not shown in the figure) in accordance with these signals.

On the other hand, when turned off, the power switch 45 connects lines w17, w18 and w19 to lines w14, w15 and w16 respectively. By this configuration, a video signal as input from the video jack 81V, an L channel audio signal as input from the audio jack 81L and an R channel audio signal as input from the audio jack 31R are given to the AV jack 83. Accordingly, the video signal and audio signals which are input from the jacks 81V, 81L and 81R are transmitted to the television monitor 5 from the AV jack 83 through the AV cable 9. As thus described, when the power supply switch 45 is turned off, it is possible to output the video signal and audio signals which are input from an external device through the jacks 81V, 81L and 81R to the television monitor 5.

The switching regulator 77 receives the power supply voltage Vcc0 from the power supply circuit 79 through the line w54 when the power supply switch 45 is turned on, and generates a ground potential GND and the power supply voltage Vcc1 on the lines w50 and w22 respectively. On the other hand, when the power supply switch 45 is turned off, the switching regulator 77 does not receive the power supply voltage Vcc0, and thereby it does not generate the power supply voltage Vcc1.

The internal power supply voltage generation circuit 75 generates power supply voltages Vcc2, Vcc3 and Vcc4 respectively on the lines w23, w24 and w25 from the ground potential GND and the power supply voltage Vcc1 as supplied from the switching regulator 77. The line w22 is connected to the terminals T7 and T8 of the connector 32; the line w23 is connected to the terminals T11 and T12 of the connector 32; the line w24 is connected to the terminals T15 and T16 of the connector 32; and the line w25 is connected to the terminals T18 and T19 of the connector 32. The respective voltages are determined such that Vcc0>Vcc1>Vcc2>Vcc3>Vcc4. Incidentally, when the power supply switch 45 is turned off, the power supply voltage Vcc1 is not generated, and thereby the power supply voltages Vcc1, Vcc2, Vcc3 and Vcc4 are not supplied to the cartridge 3 through the connector 32.

The audio amplifier 73 amplifies the R channel audio signal “AR1” as input through the line w11 which is connected to the terminal T21 and the L channel audio signal “AL1” as input through the line w10 which is connected to the terminal T20, and outputs the R channel audio signal “AR2” and L channel audio signal “AL2” which are amplified respectively to the lines w13 and w12. The line w9 for inputting the video signal “VD” to the power supply switch 45 is connected to the terminal T23 of the connector 32.

The lines w9, w12 and w13 are covered by a cylindrical ferrite 87 in order not to radiate electromagnetic waves therefrom.

The IR receiver circuit 71 digital demodulates the digital modulated infrared signal as received, and outputs digital demodulated signal to the line w8. The line w8 is connected to the terminal T17 of the connector 32. By means of this IR receiver circuit 71, an infrared signal from infrared wireless remote controller 305 is received.

Key block 69 includes the cancel key 39, arrow key 37a through 37d and decision key 35 as described above, and furthermore includes a shifting register which is not shown in the drawings. This shift register serves to convert parallel signals, which are input from the respective keys 39, 37a to 37d and 35 and a terminal TE7 described below, into serial signals, and output the serial signals to the line w3. This line w3 is connected to the terminal T6 of the connector 32. In addition, the key block 69 is given a clock signal through the line w5 which is connected to the terminal T10 and a control signal through the line w4 which is connected to the terminal T9.

The crystal oscillator circuit 67 oscillates a clock signal at a predetermined frequency (for example, 3.579545 MHz), and supplies the clock signal to the line w2. The line w2 is connected to the terminal T3 of the connector 32.

The reset switch 43 outputs a reset signal, which is used for resetting the system, to the line w1. The line w1 is connected to the terminal T4 of the connector 32.

The extension connector 63 is provided with first to ninth terminals (referred to as terminals TE1 to TE9 in the following description). The terminals TE2, TE4 and TE6 are connected to the terminals T13, T14 and T5 of the connector 32 respectively through the extension connector peripheral circuit 65. Accordingly, signals can be input from and output to an external device connected to the extension connector 63 through the terminals TE2, TE4 and TE6. The lines w4 and w5 are connected to the terminal TE 9 and TE 8 respectively. Accordingly, the same clock signal as supplied to the key block 69 can be supplied through the terminal TE8 to the external device connected to the extension connector 63, and the same control signal as supplied to the key block 69 can be supplied through the terminal TE9 to the external device.

The terminals TE3 and TE5 are supplied respectively with the power supply voltages Vcc1 and Vcc2 through the extension connector peripheral circuit 65. Accordingly, the power supply voltages Vcc1 and Vcc2 can be supplied to the external device connected to the extension connector 63 through the terminals TE3 and TE5. The terminal TE1 is grounded. The terminal TE7 is connected to a predetermined input terminal of the above shift register included in the key block 69 through the extension connector peripheral circuit 65.

FIG. 6 is a block diagram showing the internal configuration of the cartridge 3. As shown in FIG. 6, the cartridge 3 includes a high speed processor 91, a memory 93, a serial flash ROM 308, a RF module 310, the terminals t1 to t24, an address bus 95, a data bus 97 and an amplitude setting circuit 99. The amplitude setting circuit 99 includes resistors 101 and 103. The memory 93 serves as the ROM 306 shown in FIG. 4.

The high speed processor 91 includes a reset input port /RESET for inputting a reset signal, a clock input port XT for inputting a clock signal “SCLK2”, input/output ports (I/O ports) IO0 to IOn (“n” is a natural number, for example, n=23) for inputting/outputting data, analog input ports AIN0 to AINk (“k” is a natural number, for example, k=5) for inputting analog signals, audio output ports AL and AR for outputting audio signals “AL1” and “AR1”, a video output port VO for outputting a video signal “VD”, control signal output ports for outputting control signals (for example, a chip enable signal, an output enable signal, a write enable signal and so on), a data bus, and an address bus. The memory 93 includes an address bus, a data bus, and control signal input ports for inputting control signals (for example, a chip enable signal, an output enable signal, a write enable signal and so forth). The memory 93 may be, for example, a ROM (read only memory), a flash memory, or any appropriate memory.

The control signal output ports of the high speed processor 91 are connected to the control signal input ports of the memory 93. The address bus of the high speed processor 91 and the address bus of the memory 93 are connected to the address bus 95. The data bus of the high speed processor 91 and the data bus of the memory 93 are connected to the data bus 97. In this case, the control signal output ports of the high speed processor 91 include an OE output port for outputting an output enable signal, a CE output port for outputting a chip enable signal, a WE output port for outputting a write enable signal, and so forth. Also, the control signal input ports of the memory 93 include an OE input port connected to the OE output port of the high speed processor 91, a CE input port connected to the CE output port of the high speed processor 91, a WE input port connected to the WE output port of the high speed processor 91, and so forth.

When receiving the chip enable signal, the memory 93 recognizes that it is selected as the access destination to output a data signal in response to an address signal and the output enable signal which are given substantially at the same time as the chip enable signal. The address signal is input to the memory 93 through the address bus 95 while the data signal is input to the high speed processor 91 through the data bus 97. Also, when receiving the chip enable signal, the memory 93 recognizes that it is selected as the access destination to receive and write a data signal in response to an address signal and the write enable signal which are given substantially at the same time as the chip enable signal. The address signal is input to the memory 93 through the address bus 95 while the data signal is input to the memory 93 from the high speed processor 91 through the data bus 97.

The serial flash ROM 308 is connected to I/O ports IO0 and IO1 of the high speed processor 91, which are used for inputting a clock signal and reading and writing data from the high speed processor 91. Also, the RF module 310 is connected to the I/O ports 102, 103 and 104 of the high speed processor 91, which are used for inputting the clock signal, inputting and outputting data and control signals respectively.

When the cartridge 3 is installed into the adapter 1, the terminals t1 to t24 are connected to the terminals T1 to T24 of the connector 32 of the adapter 1 in a one-to-one correspondence. The terminals t1, t2, t22 and t24 are grounded. The terminal t3 is connected to the amplitude setting circuit 99. Namely, the resistor 101 of the amplitude setting circuit 99 is connected to the terminal t3 at one terminal thereof, and connected to the clock input port XT of the high speed processor 91 and one terminal of the resistor 103 at the other terminal thereof. The other terminal of the resistor 103 is grounded. Namely, the amplitude setting circuit 583 is a resistive potential divider.

The clock signal “SCLK1” generated by oscillation of the crystal oscillator circuit 67 of the adapter 1 is input through the terminal t3 to the amplitude setting circuit 99 which then generates a clock signal “SCLK2” having an amplitude smaller than the clock signal “SCLK1” and outputs the clock signal “SCLK2” to the clock input port XT. In other words, the amplitude of the clock signal “SCLK2” is set to a value which is determined by the ratio between the resistor 101 and the resistor 103.

The terminal t4 is connected to the reset input port /RESET of the high speed processor 91. Also, one terminal of the resistor 105 and one terminal of the capacitor 107 are connected to the line through which the reset input port /RESET is connected to the terminal t4. The other terminal of the resistor 105 is supplied with the power supply voltage Vcc3, and the other terminal of the capacitor 107 is grounded.

The terminals t5, t13 and t14 are connected respectively to the I/O ports IO12, IO13 and IO14 of the high speed processor 91. Accordingly, the high speed processor 91 can output signals to and input signals from the external device connected to the extension connector 63 of FIG. 5 through the terminals t5, t13 and t14.

The power supply voltage Vcc1 is supplied from the terminals t7 and t8. The power supply voltage Vcc2 is supplied from the terminals t11 and t12. The power supply voltage Vcc3 is supplied from the terminals t15 and t16. The power supply voltage Vcc4 is supplied from the terminals t18 and t19. The power supply voltage Vcc2 is supplied to the analog circuitry of the high speed processor 91 while the power supply voltage Vcc3 is supplied to the digital circuitry of the high speed processor 91.

The terminals t6, t9, t10 and t17 are connected respectively to the I/O ports IO15, IO16, IO17 and IO18 of the high speed processor 91. Accordingly, the high speed processor 91 can receive a signal output from the key block 69 through the terminal t6. Also, the high speed processor 91 can output a control signal to an external device connected to the extension connector 63 and the key block 69 through the terminal t9. Furthermore, the high speed processor 91 can supply a clock signal to an external device connected to the extension connector 63 and the key block 69 through the terminal t10. Still further, the high speed processor 91 can receive the output signal of the IR receiver circuit 71 through the terminal t17.

The terminals t20 and t21 are connected to the audio output ports AL and AR of the high speed processor 91. The terminal t23 is connected to the video output port VO of the high speed processor 91. Accordingly, the high speed processor 91 can output the audio signals “AL1” and “AR1” to the audio amplifier 73 of the adapter 1 through the terminals t20 and t21, and output the video signal “VD” to the power supply switch 45 of the adapter 1 through the terminal t23.

The cartridge 3 is provided with a shield member 113. By virtue of the shield member 113, electromagnetic waves can be prevented, as much as possible, from leaking from the high speed processor 91 and the like as external radiation.

Next, the internal configuration of the high speed processor 91 will be briefly explained. Although not shown in the figure, the high speed processor 91 includes a CPU (central processing unit), a graphics processor, a sound processor and a DMA controller and so forth, and in addition to this, includes an A/D converter for receiving analog signals, and an input/output control circuit for receiving input signals such as key operation signals and infrared signals and giving the output signals to external devices.

The CPU takes control of the entire system and performs various types of arithmetic operations in accordance with a program stored in the memory 93.

The graphics processor constructs graphics data on the basis of data stored in the memory 93, and outputs a video signal “VD” which is generated on the basis of the graphics data and compatible with the television monitor 5.

In this case, the graphics data is generated by synthesizing a background screen(s), a sprite(s) and a bitmap screen. The background screen is provided in the form of a two-dimensional array consisting of rectangular pixel sets, and has a size covering the entirety of the screen of the television monitor 5. There are a first background screen and a second background screen respectively prepared as the background screen for showing depths. The sprite consists of a rectangular pixel set which can be relocated in any position of the screen of the television monitor 5. The bitmap screen consists of a two-dimensional pixel array, and the size and position thereof can be freely designated.

In addition to this, the high speed processor 91 includes a pixel plotter which is not shown in the figure but can perform drawing operations with individual pixels.

The sound processor converts data stored in the memory 93 into sound data, and generates and outputs the audio signals “AL1” and “AR1” on the basis of the sound data. The sound data is synthesized by pitch conversion and amplitude modulation of PCM (pulse code modulation) data serving as the base data of tone quality. For the amplitude modulation, an envelope control function for reproducing waveforms of a music instrument is provided in addition to a volume control function performed in response to an instruction of the CPU.

In addition to this, the high speed processor 91 is provided with an internal memory (not shown in the figure) which is used as a working area, a counter area, a register area, a temporary data area, a flag area and/or the like area.

[Communication Between Systems]

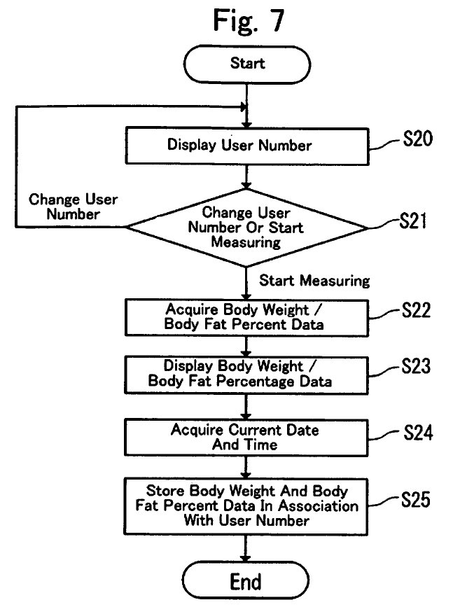

FIG. 7 is a flow chart showing the process of measuring a body weight by the MCU 320 of the body weight scale 300 of FIG. 4. As shown in FIG. 7, in step S20, the MCU 320 displays the user number in the display unit 302. If there is an instruction to change the user number when the user manipulates the switch unit 319 in step S21, the MCU 320 proceeds to step S20 in which the user number as changed is displayed. On the other hand, when the user gets on the body weight scale so that an ON signal is input from the detection switch of the switch unit 319, the MCU 320 proceeds to step S22.

In step S22, the MCU 320 acquires the body weight and body fat percentage data from the body weight/fat measuring unit 318. In step S23, the MCU 320 displays the body weight value and the body fat percentage in accordance with the body weight/body fat percentage data as acquired on the display unit 302. In step S24, the MCU 320 acquires the current date and time data from the real time clock unit 316. In step S25, the MCU 320 stores the body weight and body fat percentage data and the current date and time data, which are acquired, in the EEPROM 314 in association with the user number which is currently selected.

By the way, if a predetermined time elapses without any manipulation, the body weight scale 300 enters a stand-by mode. In the stand-by mode, the body weight scale 300 performs communication processing in response to the interrupt from the real time clock unit 316 which is repeatedly issued at predetermined time intervals (for example, one minute). In what follows, details will be explained.

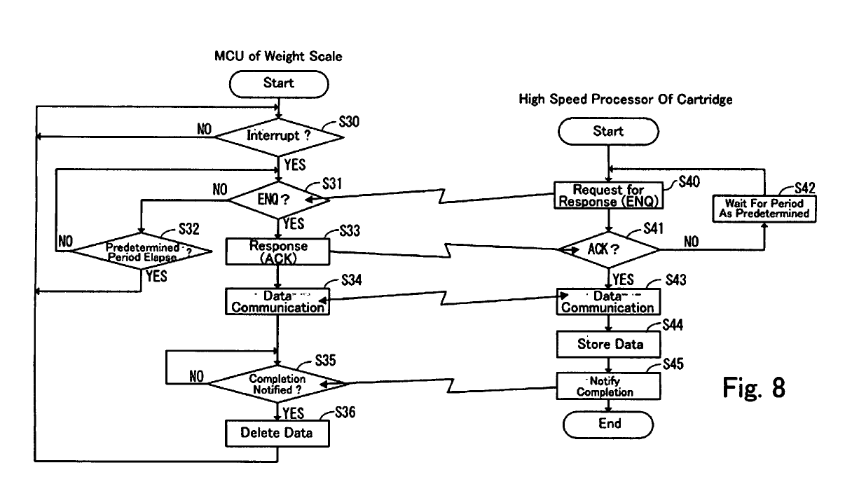

FIG. 8 is a communication protocol diagram between the cartridge 3 and the body weight scale 300 of FIG. 4. FIG. 8 shows the process of the MCU 320 of the body weight scale 300 in the stand-by mode and the process of the high speed processor 91 after turning on the power supply switch 8 of the adapter 1 into which the cartridge 3 is inserted. As shown in FIG. 8, if there is an interrupt from the real time clock unit 316 in step S30, the MCU 320 of the body weight scale 300 proceeds to step S31 otherwise repeats the same step S30. As has been discussed above, the real time clock unit 316 issues an interrupt at predetermined time intervals.

If there is an enquiry (ENQ) from the high speed processor 91 of the cartridge 3 inserted into the adapter 1 in step S31, the MCU 320 proceeds to step S33 otherwise proceeds to step S32. In this case, since the enquiry from the high speed processor 91 is received by the RF module 312, the MCU 320 communicates with the RF module 312 to determine whether or not there is an enquiry.

In step S32, the MCU 320 determines whether or not a predetermined time elapses, and if it elapses the process proceeds to step S30 otherwise proceeds to step S31.

On the other hand, in step S33, since an enquiry is transmitted from the high speed processor 91 (the “Y” branch from step S31), the MCU 320 returns an acknowledgement (ACK) to the high speed processor 91. In this case, the MCU 320 transmits the acknowledgement to the high speed processor 91 through the RF module 312.

In step S34, the MCU 320 performs data communication with the high speed processor 91. In this case, the MCU 320 performs data communication through the RF module 312. In this data communication, the MCU 320 first transmits, to the high speed processor 91, the date and time data saved therein at this time, and then transmits, to the high speed processor 91, the body weight and body fat percentage data and the date and time data of the respective users stored in the EEPROM 314.

In step S35, the MCU 320 confirms whether or not a data reception completion notification is transmitted from the high speed processor 91, and if the completion notification is transmitted, the process proceeds to step S36 otherwise proceeds to step S35. In this case, since the completion notification from the high speed processor 91 is received by the RF module 312, the MCU 320 communicates with the RF module 312 to confirm whether or not there is the completion notification. In step S36, the MCU 320 deletes the body weight and body fat percentage data and the date and time data stored in the EEPROM 314, and proceeds to step S30.

On the other hand, when the cartridge 3 is inserted into the adaptor 1 and the power supply switch 8 is turned on, the high speed processor 91 sends an enquiry to the MCU 320 of the body weight scale 300 in step S40. In this case, the high speed processor 91 sends the enquiry to the MCU 320 through the RF module 310.

If an acknowledgement (ACK) is transmitted from the MCU 320 in step S41, the high speed processor 91 proceeds to step S43 otherwise proceeds to step S42. In this case, since the acknowledgement is received by the RF module 310, the high speed processor 91 communicates with the RF module 310 to confirm whether or not there is an acknowledgement.

In step S42, the high speed processor 91 waits for a predetermined time, and thereafter proceeds to step S40. On the other hand, in step S43, since the acknowledgement is sent from the MCU 320 (the “Y” branch from step S41), the high speed processor 91 performs data communication with the MCU 320. In this case, the high speed processor 91 performs the data communication through the RF module 310. In this data communication, the high speed processor 91 acquires the body weight and body fat percentage data and the date and time data of the respective users transmitted from the MCU 320.

In step S44, the high speed processor 91 stores the body weight and body fat percentage data and the date and time data of the respective users in the serial flash ROM 308. In step S45, the high speed processor 91 transmits a completion notification of data reception to the MCU 320. In this case, the high speed processor 91 transmits the completion notification to the MCU 320 through the RF module 310.

[Examples of Display Screen]

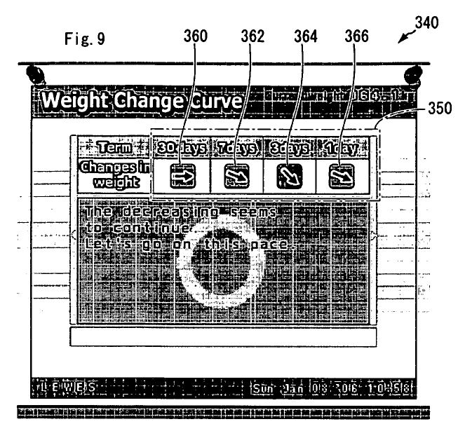

The system in accordance with the present embodiment performs the body weight management while displaying a variety of images on the television monitor 5. FIG. 9 to FIG. 13 show several examples of a screen as displayed on the television monitor 5 on the basis of the video signal VD which is generated by the high speed processor 91.

FIG. 9 shows a display screen 340 which displays the change tendency of body weight when this health management program starts running. Referring to FIG. 9, this screen 340 includes a body weight tendency display area 350 in which the change tendency of body weight in the past is displayed.

The body weight tendency display area 350 includes a first arrow indication 360 which indicates the change tendency of body weight for past 30 days, a second arrow indication 362 which indicates the change tendency of body weight for past 7 days, a third arrow indication 364 which indicates the change tendency of body weight for past 3 days, and a fourth arrow indication 366 which indicates the change tendency of body weight on the current day. These arrow indications are arranged in the lateral direction at the same time. Each of these arrow indications is a symbol which indicates the change tendency of body weight by its direction, and has a meaning which is briefly described as follows. An arrow pointing to the right (for example, the first arrow indication 360) indicates that the body weight tended to level off in the corresponding period. An arrow pointing to the lower right (for example, the second arrow indication 362) indicates that the body weight tended to decrease in the corresponding period. An arrow pointing to the upper right (no example is shown in FIG. 9) indicates that the body weight tended to increase in the corresponding period. Of these arrow indications 360, 362, 364 and 366, the third arrow indication 364 and the second arrow indication 362 serve as examples illustrating that the inclination of the arrow indicates the degree of tendency to increase or decrease. Also, although not illustrated in FIG. 9, these arrow indications 360, 362, 364 and 366 are displayed with different colors in accordance with the change tendency of body weight. Namely, the display color is changed such that, for example, green is used if the body weight tended to level off, blue is used if the body weight tended to decrease, red is used if the body weight tended to increase, otherwise an intermediate color therebetween is used corresponding to the tendency therebetween. This display makes it possible for the user to know the change tendency of body weight in the long, medium and short term with reference to the inclination of the arrow, and intuitively and easily understand the tendency with reference to the color. In addition, since this screen 340 is necessarily displayed at the start of the process, it is possible to make a strong impression on the user.

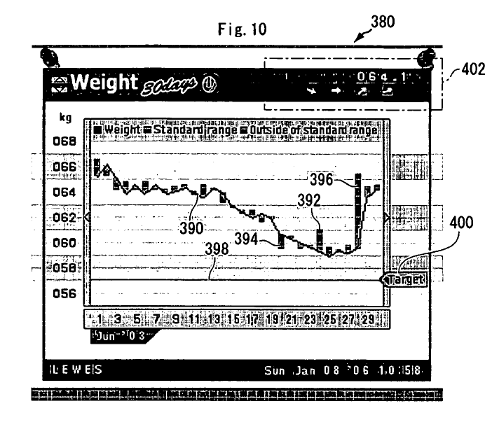

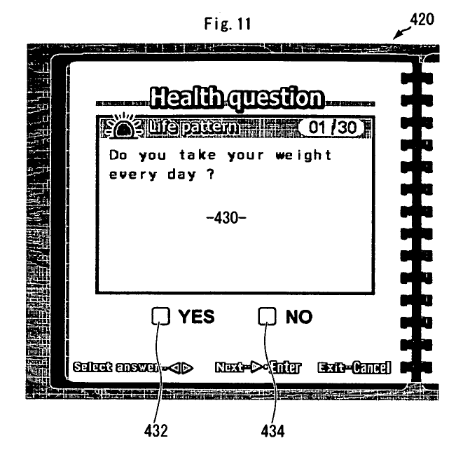

FIG. 10 shows a chart display screen 380 indicating the body weight change in accordance with the present embodiment. With reference to FIG. 10, the graphs representing the body weight change shown in the chart display screen 380 include a combination of a line graph 390 and a vertical bar graph indicated by reference numbers 392, 394 and 396. In the case of the present embodiment, it is assumed that the user measures own body weight twice a day, i.e., in the morning (just after the wake-up time) and night (just before the bedtime). In the example shown in FIG. 10, the line graph 390 is plotted to represent the body weights measured in the morning of every day from 30 days before up to the current day. Namely, the abscissa of the chart shows the number of days counted from the starting day, i.e., 30 days before, and the ordinate of the chart shows the body weight (kg).

The vertical bars 392, 394, 396 and the like represent the differences between the body weight in the night and the body weight in the morning of the same day by the lengths thereof. That is, the vertical bars show how much the body weight in the night increases relative to the body weight in the morning of the same day within the same day. In the case of the present embodiment, when the body weight in the night increases from the body weight in the morning, a vertical bar is plotted in order to extend upwardly from the plot of the line graph 390 corresponding to the current day. On the other hand, when the body weight in the night decreases from the body weight in the morning, a vertical bar is plotted in order to extend downwardly from the plot of the line graph 390 corresponding to the current day. There is an advantages that the body weight change in the current day can be easily understand from the vertical bars 392, 394 and 396. Also, in the usual case, the body weight takes the lightest value just after the wake-up time, and takes a value heavier than the morning weight in the night (after supper). This differential body weight varies between individuals, however, it is possible to determine a reasonable value. If the differential body weight is larger than the reasonable value, it is assumed that the user ate too much or took too little exercise in that day. Accordingly, in the case of the present embodiment, if the difference between the body weight in the night and the body weight in the morning is larger than the value which can be considered reasonable, the vertical bar is displayed by red, otherwise (inclusive of the day in which the body weight in the night is smaller than in the morning) the vertical bar is displayed by blue. As described above, there is an advantage that, by the use of the vertical bar representing the differential weight and being colored to distinctively indicate whether or not the differential weight is usual, it is possible for the user to intuitively and easily understand whether or not the body weight change in the current day is reasonable in the same manner as in FIG. 9.

More specifically speaking, in the case of the present embodiment, if the difference between the body weight in the night and the body weight in the morning is larger than 0.83% of the body weight in the morning, the vertical bar is displayed by red, otherwise the vertical bar is displayed by blue. Meanwhile, in the case of the present embodiment, 0.83% of the body weight is used as a default value of the threshold value. However, as described below, this value can be changed by manually inputting. Alternatively, the threshold value may be automatically set up, for example, by preparing a table of threshold values in units of kilograms in association with gender, body height and age, and looking up an appropriate threshold value in the table in accordance with the gender, body height and age of the user.

In the upper right corner of this screen 380, a body weight tendency display area 402 is provided as a miniature version of the body weight tendency display area 350 shown in FIG. 9. There are arrows which are similar to those shown in FIG. 9 and separated by color. By this display, for both the line graphs and the vertical bar graph, there is an advantage that the understanding of the change tendency of body weight becomes furthermore easy. Also, while confirming the change tendency of body weight in the past by this display, the user can be review various information related to this change tendency and make an appropriate judgment on the basis of the information relating to own body weight.

Incidentally, when the system operates in a “diet mode” to be described below, a target indication 400 and a target line 398 are displayed in order to indicate a target body weight of diet as shown in FIG. 10. Since the line graph 390 and the target line 398 are displayed in the same screen in this manner, the user can easily compare them and be encouraged to diet.

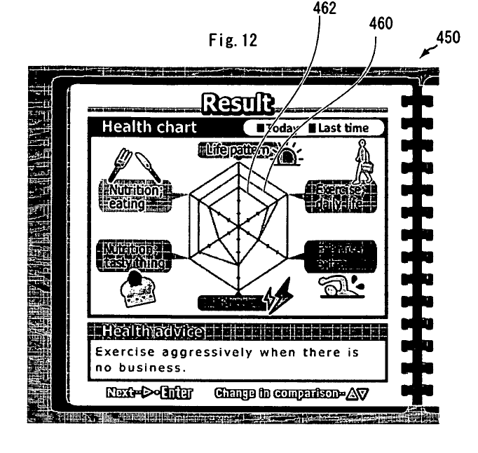

FIG. 11 shows a checklist screen 420 prepared for the user to answer questions about health by the system in accordance with the present embodiment. This checklist screen 420 can be selected and opened from the menu of the chart screen 380 (launched by the enter key) as shown in FIG. 10. In the checklist screen 420, predetermined 30 questions about health are successively displayed in a question area 430 where the user selects a YES button 432 or a NO button 434 to answer these questions. After selecting either of these buttons by the left and right keys, an answer can be input by pressing the enter key. These 30 questions are classified into six genres of five questions. After the user answers all the 30 questions with “YES” or “NO”, the result is displayed in a radar chart 450 of FIG. 12. In the display of the result, an advice comment is displayed in addition to the radar chart.

Meanwhile, the six genres includes (1) life pattern, (2) nutrition (eating), (3) nutrition (tasty thing), (4) stress, (5) exercise (daily life) and (6) exercise (sports).

The radar chart 450 of FIG. 12 shows the result of answers from the user to the five questions (30 questions in total) belonging to each of the six genres as described above. In the case of the present embodiment, the radar chart 462 corresponding to the result of the current answers is displayed in the radar chart 450 in contrast with the radar chart 460 corresponding to the previous answers.

By preparing the checklist screen 420, asking the user to answer questions, displaying the current result in contrast with the previous result, the user can examine own habit in relation to health and more effectively perform the health management.

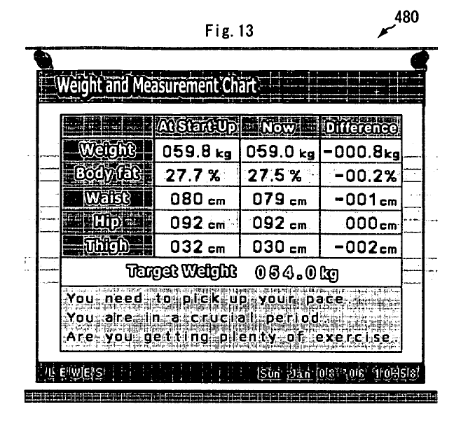

FIG. 13 shows a progress/achievement display screen 480 displayed after daily body weight measurement in the diet mode to be described below. In the diet mode, the current body weight, body fat percentage and the like are displayed as well as those just before diet in contrast with each other as illustrated in FIG. 13. Also, a message according to the contrasting result is displayed in the lower area of the progress/achievement display screen 480.

### [Program Flow Chart]

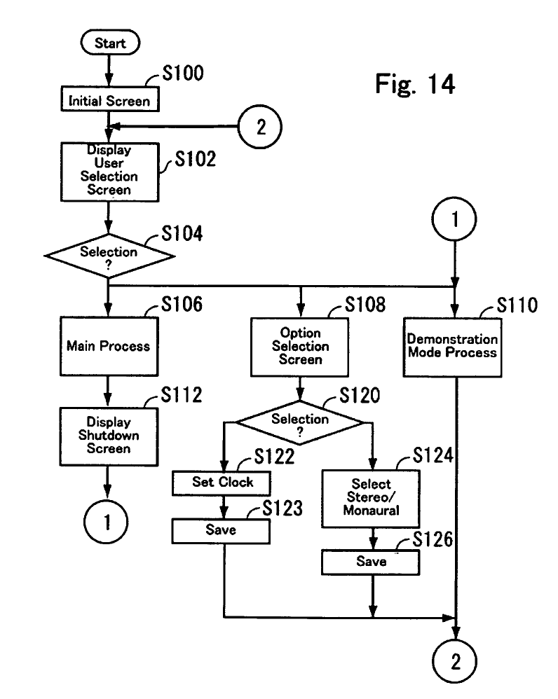

FIG. 14 is a flowchart showing the main routine of a program stored in the cartridge 3 for realizing this system. As shown in FIG. 14, when power is applied, an initial screen is displayed in step S100. This initial screen is not a single screen but a multiple screen having a plurality of consecutive screens which are displayed for a predetermined time. For example, the display screen of the manufacture's name of the program, the title screen of the program and the title screen of the product are displayed in this order. In the case of the present embodiment, these screens are displayed in this order, and then the system waits for user's selection while displaying the product title screen. Namely, in step S102, the list of registered users is displayed in the product title screen, and the system waits until the user selects an item of the list. In this case, there is also displayed a blank space as a user name, and when this blank space is selected the user is treated as a new user. “Guest” is also a user name as displayed. If the user is a guest, only the measurement of body weight is possible. Furthermore, an optional selection process can be selected here.

When a certain selection is made, the user name designated in step S104 is examined. If the user has already been registered and a password is set, a password input screen is displayed. If a password is not set or if a password is set and the password is correctly input, the main process for managing the body weight of this user is performed in step S106. If the optional selection process is selected, the processing proceeds to step S108 in which the screen of selecting an option is display for making settings associated with the body weight management. Meanwhile, if there is no operation by the user for a predetermined time in step S102, such no operation is detected in step S104, and the process proceeds to step S110 in which a demonstration mode screen is displayed. In the demonstration mode screen, a predetermined demonstration is displayed. While the game apparatus can display a screen in which a demonstration of the game is performed, the health management support system such as that of the present embodiment cannot perform such demonstration. Thereby, in the case of the present embodiment, a motion screen is displayed for explaining how to read various graphs which are displayed for managing the body weight. The adapter 1 can be powered off in the demonstration mode. When some operation is made on the cartridge 3 or the remote control 305 in the demonstration mode in step S110, the process is returned to step S102.

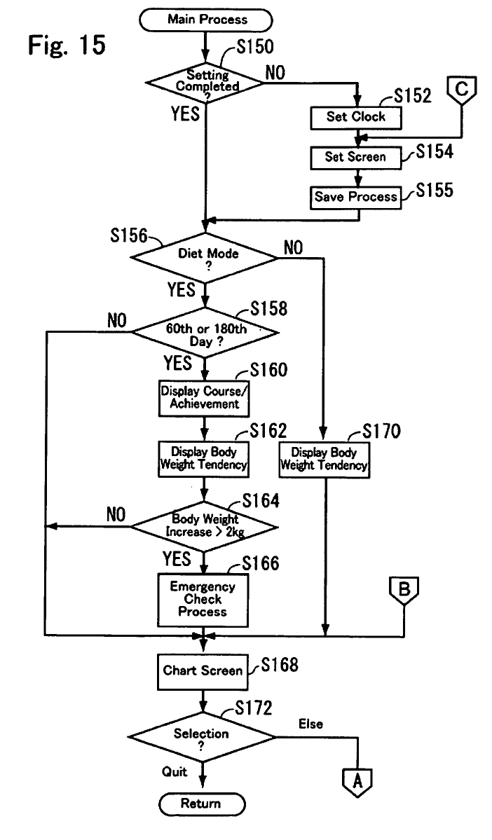

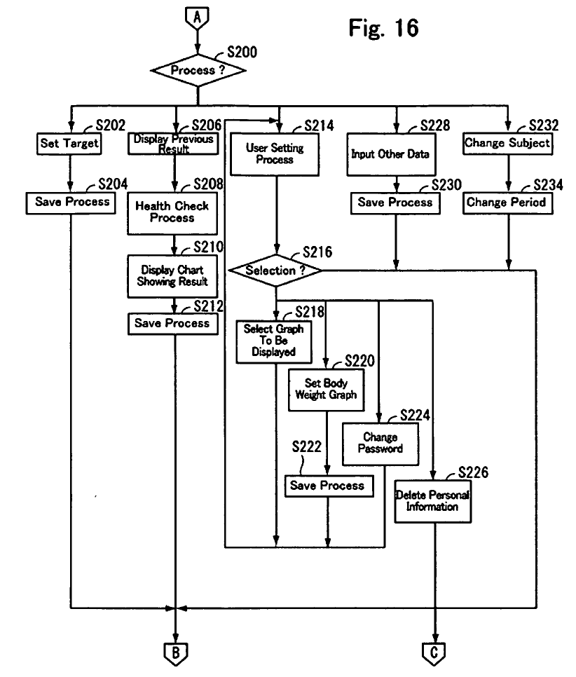

In step S106, the main process for managing the body weight of the selected user is performed. The main process will be described below in detail with reference to FIG. 15 and FIG. 16. If there is no operation for five minutes during performing the main process, the process proceeds to step S112 in which the selection of the user is made invalid (log-out processing), and proceeds to step S110 in which the demonstration mode screen is displayed. Also, when the cancel button is pressed by the user during performing the main process, the process proceeds to step S112 in which the selection of the user is made invalid (log-out processing), and after displaying a shutdown screen, the process proceeds to step S110 in which the demonstration mode screen is displayed.

If the optional selection screen is selected in step S102, the process proceeds from step S104 to step S108 for displaying the optional selection screen. In the optional selection screen in step S108, it is possible to set a clock and select stereo audio or monaural audio as optional operations.

Namely, if setting the clock is selected in the optional selection screen in step S108, the date and time are set by the user in step S122, and thereafter the process proceeds to step S123 in which the setting is saved and returned to step S102. The date and time which are set in this process are transmitted to the body weight scale 300 by wireless communication, and the date and time of the body weight scale 300 are set also to these values. If the process of selecting stereo audio or monaural audio is selected, stereo audio or monaural audio is selected in step S124. The result of the selection is saved in step S126. Thereafter, the process is returned to step S102.

FIG. 15 and FIG. 16 show a flow chart of the main process performed in step S106 of FIG. 14. By this process, the daily body weight management of the user can be performed. First, in step S150, it is determined whether or not the settings of time, user name and the like have been completed.

If completed, the process proceeds to step S156. If not completed yet, the process proceeds to step S152. In step S152, time is set. Thereafter, the settings of user data are performed in step S154. The user data to be set includes user name (manually input), gender (manually input), birthday (manually input), system of unit as selected (pound/inch system, or kilogram/centimeter system), body height (manually input), target body weight, intensity of daily activity (daily activity index), password, subject of graph as selected, and so forth. After completing the settings, the BMI, degree of obesity, recommended body weight and basal metabolic amount of this user are automatically calculated. Also, the body weights of the respective users are automatically set on the basis of the result of measurement.

The BMI, recommended body weight, basal metabolic amount, energy needed per day, standard value of basal metabolic rate are automatically calculated on the basis of the following equations.

BMI=body weight (kg)÷body height (m)÷body height (m) recommended body weight=height (m)×height (m)×22 basal metabolic amount per day (female)=665+(9.6×body weight (kg))+(1.7×height (cm))−(7.0×age) basal metabolic amount per day (male)=66+(13.7×body weight (kg))+(5.0×height (cm))−(60.8×age) energy needed per day=basal metabolic amount×daily activity index standard value of basal metabolic rate=basal metabolic amount÷body weight (kg) One of the following comments is displayed in accordance with the value of BMI.

TABLE 1

| BMI | Degree of Obesity | Comments |
|---|---|---|
| less than 18.5 | too slim | a little skinny |
| 18.5 to less than 25 | standard | balanced body |
| 25 to less than 30 | first degree of obesity | a little obese |
| 30 to less than 35 | second degree of obesity | tend to be obese, losing |
| | | weight is needed |
| 35 to less than 40 | third degree of obesity | obese, please lose weight |
| 40 or higher | fourth degree of obesity | extremely obese, please |
| | | talk to a doctor and lose |
| | | weight |

TABLE 2

| | Intensity of Daily Activity | | | |
|---|---|---|---|---|
| | 1 (low) | 2 (a little low) | 3 (normal) | 4 (high) |
| Daily Activity Index | 1.3 | 1.5 | 1.7 | 1.9 |
| Daily Life | walking (promenade, shopping | walking for two hours or | walking or cycling for a hour | hard workout for a hour |
| Style | and so forth) for a hour or | thereabout a day to commute, | or thereabout a day, farm or | or thereabout a day, |
| | thereabout a day, reading, | work and the like, service | fishery work for a hour or | conveyance of lumber, |
| | study, talking, TV viewing | to customers, housework and | thereabout a day, in the case | farm work in rush season |
| | and so forth | the like | of persons with intensity of | and the like |
| | | | daily activity of 2 | |
| Main Job | clerk general | service job, housework on an | farm work, construction work, | farm work in rush season, |
| | | errand, most office workers | so-called physical work | sport for pay and the like |
| | | belong to this class | | |

TABLE 3
Standard Value of Basal Metabolic Rate and Basal
Metabolic Amount as Separated by Age Brackets

| | | |
|---|---|---|
| | Standard Value of | |
| | Basal Metabolic Rate | Basal Metabolic |
| Age | (kcal/kg/day) | Amount (kcal/day) |

| (years) | male | female | male | female |
|---|---|---|---|---|
| 1-2 | 61.0 | 59.7 | 700 | 700 |
| 3-5 | 54.8 | 52.2 | 900 | 860 |
| 6-8 | 44.3 | 41.9 | 1090 | 1000 |
| 9-11 | 37.4 | 34.8 | 1290 | 1180 |
| 12-14 | 31.0 | 29.6 | 1480 | 1340 |
| 15-17 | 27.0 | 25.3 | 1610 | 1300 |
| 18-29 | 24.0 | 23.6 | 1550 | 1210 |
| 30-49 | 22.3 | 21.7 | 1500 | 1170 |
| 50-69 | 21.5 | 20.7 | 1350 | 1110 |
| 70 or older | 21.5 | 20.7 | 1220 | 11010 |

In step S156, it is determined whether or not the diet mode is selected by the user who is using the system. The diet mode is an operation mode in which after setting a period of 180 days the user goes on diet until a target body weight is reached. In the diet mode, while calculating the basal metabolic amount, various judgments and messages are displayed in order to go on diet without upsetting the health on the basis of the body weight change. Meanwhile, in the diet mode, the specification is such that the target cannot be set lower than or equal to 90% of the current body weight.

If the diet mode is selected, the process proceeds to step S158, otherwise proceeds to step S170. In step S170, the body weight tendency screen is displayed, and the process proceeds to step S168. The body weight tendency screen is the screen 340 shown in FIG. 9.

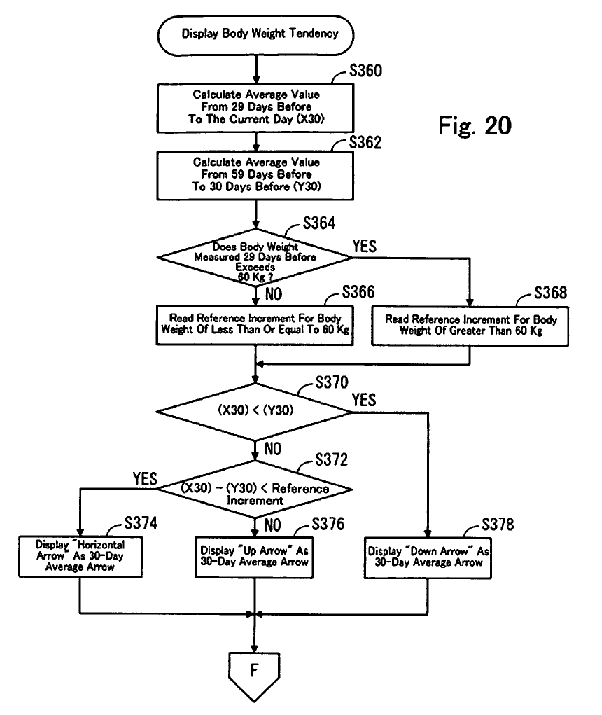

The body weight tendency is displayed in the body weight tendency screen in accordance with the following formula. Namely, after calculating the differences of the last body weight from the body weights measured previously, 3 days before, 7 days before and 30 days before respectively, the arrow indications 360 and the like shown in FIG. 9 are determined on the basis of the calculation results. Meanwhile, these body weights are compared by the use of the moving averages thereof. In other words, when comparing the body weight measured 3 days before and the last body weight, the three day moving averages thereof are compared. When comparing the body weight measured 7 days before and the last body weight, the seven day moving averages thereof are compared. When comparing the body weight measured 30 days before and the last body weight, the thirty day moving averages thereof are compared. However, when comparing the body weight previously measured and the last body weight, the comparison is made only with one differential day, and thereby the measurement values of the current day and the previous day are compared. The judgment of this body weight tendency will be described below with reference to FIG. 20 through FIG. 23.

Arrows are decided in accordance with the following determination criteria.

TABLE 4

Base Body Weight (kg)

| Number of | up to | up to | up to | up to | up to | up to |
|---|---|---|---|---|---|---|
| Days | 50 | 60 | 80 | 100 | 120 | 140 |
| one day | 1.0 | 1.0 | 1.5 | 1.5 | 1.5 | 1.5 |
| three days | 1.0 | 1.0 | 1.5 | 1.5 | 1.5 | 1.5 |
| seven days | 1.0 | 1.0 | 1.5 | 1.5 | 1.5 | 1.5 |
| thirty days | 1.5 | 1.5 | 2.0 | 2.0 | 2.0 | 2.0 |

Namely, the reference change amounts are twofold depending upon whether or not the base body weight is greater than 60 kg, and are provided for each of the units of exercise days, i.e., four units (one day, 3 days, 7 days and 30 days).

When the body weight is increasing, if the increasing amount is equal to the above value or up to the above value a laterally pointing arrow is displayed with green, and if the increasing amount exceeds the above value an arrow pointing to the upper right is displayed with red. Incidentally, the number of levels into which unvarying or increasing body weights are divided can be three rather than two such that three types of arrow inclination are used. Furthermore, four or more levels are provided to use four or more types of arrow inclination. Also, in the case of the present embodiment, if the result of the comparison indicates that the current body weight decreases than before, a single type of arrow pointing to the lower right is displayed with blue irrespective of the amount of decrement.

While the change tendency of the body weight is indicated by an arrow in the case of the present embodiment, the present invention is not limited to this, but any other symbols can be used. For example, “.” is used when the body weight increases by an amount larger than or equal to the threshold value as described above, “Δ” is used when the body weight levels off, and “.” is used when the body weight decreases. The understanding is further facilitated by combining the above symbols with coloring. In particular, when the body weight increases, it is preferred to use red for the purpose of inviting the user's attention.

Referring again to FIG. 15, in the diet mode, it is determined whether or not the current day is the 60th or 180th day after starting diet in step S158. If the current day is the 60th or 180th day, the process proceeds to step S160, otherwise the process proceeds to step S168.

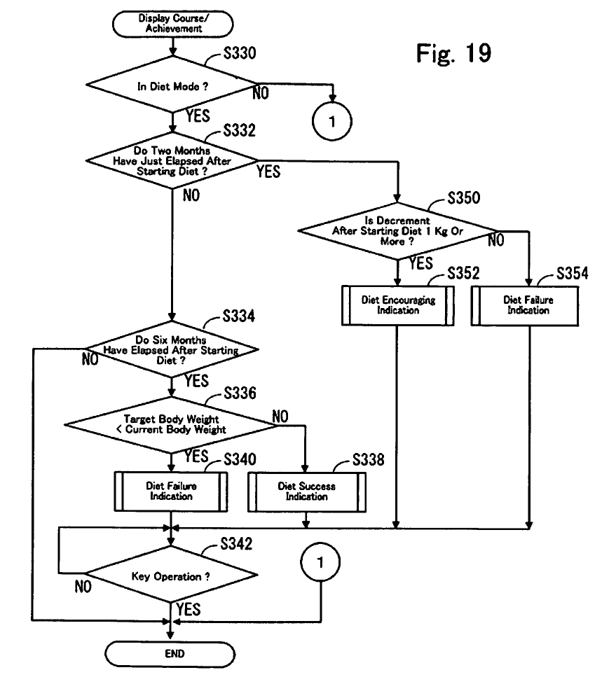

In step S160, the course of the body weight change (in the 60th day) or the achievement (in the 180th day) is displayed. If the current day is the 60th day, it is judged whether or not the diet is likely to succeed, and a message is displayed in accordance with the result of judgment. If the current day is the 180th day, it is judged whether or not the diet succeeded by comparing the body weight as measured and the target body weight, and the result of judgment is displayed. The details of this will be described below with reference to FIG. 19.

After step S160, the body weight tendency is displayed in step S162. This is the same as displayed in step S170 and corresponds to the screen 340 shown in FIG. 9.

Thereafter, it is determined whether or not the increase in the body weight after previous measurement exceeds 2 kg in step S164. If the increase in the body weight does not exceed 2 kg, the process proceeds to step S168, otherwise the process proceeds to step S166 in which an emergency check process is performed. In this case, this weight of “2 kg” is fixed, however, it is also possible to set this weight as an appropriate function of the body weight of the user. For example, this weight can be set separately for each base body weight as shown in the table 4. In the emergency check process, a problem screen is displayed as a pop-up window in the same manner as shown in FIG. 11 to ask the user to answer questions. These questions are provided in advance, separately from those shown in FIG. 11, as 30 questions for each of the groups separated by gender and three age brackets, totaling to 2×3×30=180 questions. In this case, one of the questions is selected by the use of a random number and displayed. No particular process is performed in response to the answer. However, the aim is to encourage the user to reaffirm the attitude to diet and make a better life habit by answering the questions.

Incidentally, the answers of the respective questions include advisable answers and unadvisable answers. If the user gives an advisable answer in response to a problem, this problem is excluded from the problems from which next and subsequent selection is made. The problem in response to which the user gave an unadvisable answer is included in the problems from which next and subsequent selection is made.

In step S168, the chart screen is displayed. As described above, this chart screen is the screen showing the body weight change as illustrated in FIG. 10. Meanwhile, in this chart screen, a graph other than the body weight graph can be displayed. This selection is made as the user settings to be described below. The chart screen displayed in step S168 is a base screen from which various processes can be performed. In other words, after displaying this chart screen, the high speed processor 91 waits for the instruction of the user. If there is an instruction in step S172, the process corresponding to this instruction is started. However, if there is an instruction to quit, the main process ends and returns to step S112 of FIG. 14.

In what follows, the processes which can be performed from the chart display screen are described. In this case, first, the period and subject of a graph can be changed by manipulating the up and down keys and the right and left keys. The period of the graph can be selected from among 30 days, 90 days and 360 days. The subject presented by the graph can be selected from among the changes in the body weight, the body fat percentage, taken/consumed calories, number of steps, three sizes, blood pressure as measured and basal body temperature, and so forth.

By pressing the right or left key, the dates of the graph are scrolled in the pressing direction. By manipulating “the enter key+the up and down keys”, it is possible to jump to one of four screens for performing the processes of “data input” for inputting the so-called three sizes, number of steps, blood pressure, basal body temperature and the like, “check list” for answering 30 questions as a health check, “target setting” for setting a target body weight in the diet mode, and “user setting” for performing a variety of settings about the user. As a result, one of these processes is selected. It is also possible to return to the chart screen from the jump target screen by pressing the cancel key.

FIG. 16 is a flow chart for showing the process flow performed as the result of selection in step S172. Referring to FIG. 16, it is determined which process is selected in step S200. The process which can be selected is one of the processes of “data input”, “check list”, “target setting”, “user setting”, changing the subject of graph, and changing the period of graph.

If the target setting process is selected the process proceeds to step S202, if the check list process is selected the process proceeds to step S206, if the process of inputting other data is selected the process proceeds to step S228, if the user setting process is selected the process proceeds to step S214, and if the process of changing the subject or period of graph is selected the process proceeds to step S232.

In step S202, the target body weight in the diet mode of this user is set. The value as set is saved in step S204, and the process is returned to step S168 of FIG. 15.

If the check list process is selected, the result of the previous check list process is displayed in a radar chart in step S206.

In step S208, the 30 questions prepared in advance about health are sequentially displayed to ask the user to answer the questions as a health check process. In step S210, the radar chart for the current process is created on the basis of the answer from the user, and displayed together with the previous radar chart in contrast with each other. This display example is as shown in FIG. 12. If there are available past results before the previous check (for example, in the previous month, three months before, six months before, one year before and so forth), these results can be displayed to compare with the current result. In step S212, the current result is saved. This result is also displayed in step S206 as the “previous result” when this process is selected again in the next routine. After step S212, the process is returned to step S168 (FIG. 15). The result of this check list process can be saved each time the check list process is performed.

If the user setting process is selected, the process proceeds to step S214 in which a screen for the user setting process is displayed. A variety of processes for the user can be selected from this screen. In step S216, it is determined what is selected by the user as the subject to be set. If the subject of the graph stored in step S168 is selected the process proceeds to step S218, if the setting of the body weight graph is selected the process proceeds to step S220, if the process of changing the password is selected the process proceeds to step S224, if the deletion of the user information is selected the process proceeds to step S226.

In step S218, the process of selecting the default subject of graph to be displayed is performed. This selection screen includes check boxes for selecting the subject to be displayed by a graph (body weight, body fat percentage, taken/consumed calories, number of steps, three sizes, measured blood pressure, basal body temperature and the like) and the covered period in combination. If a check box is checked, a corresponding graph is displayed in step S168 while no graph corresponding to unchecked boxes is displayed. Nevertheless, a 30 day graph showing the body weight is always displayed. Thereafter, the process is returned to step S214. Meanwhile, the information other than the body weight and the body fat percentage, which is used herein, is the information which is input and accumulated in step S228. Also, if the check boxes corresponding to the measured blood pressure and basal body temperature are unchecked, no input item corresponding thereto is displayed in the input screen.

In step S220, the detailed settings of the body weight graph are performed. With respect to the body weight graph, the threshold value can be set which is used to determine the color of the vertical bar for showing the body weight change between morning and night. The default value of the threshold value for color determination is 0.83% of the body weight as described above. However, the default value can be set to a different value also as described above. In addition, the basic measurement time can be changed. That is to say, in the case of a person having the so-called late-night life pattern, it is possible to make the setting of the body weight comparison as “comparison between previous night and next morning” rather than the usual pattern of “comparison between morning and night in the same day”. After completing the settings, the data as set is saved in step S222, and the process is returned to step S214.

In step S224, a new password is set, or an existing password is erased. Thereafter, the process is returned to step S214.

In step S226, the personal information of the currently logged-on user is erased, and the process proceeds to step 154 of FIG. 15.

Meanwhile, if the cancel key is pressed in the screen of processing user settings in step S214, it is determined that returning to the chart screen is selected in step S216, and the process proceeds to step S168 of FIG. 15.

On the other hand, if the screen for “inputting other data” in the chart screen in step S168, the process proceeds to step S228 as a result of the determination in step S200. In step S228, three sizes, number of steps, blood pressure, basal body temperature and the like are input. In step S230, the input data is saved, and the process is returned to step S168.

When the user operates the up or down key or the right or left key in the chart screen in step S168, the process proceeds to step S232. The process after step S232 is the process of changing a graph. If the up or down key is operated, the subject of the graph is changed in step S232. If the right or left key is operated, the display period of the graph is changed in step S234. Thereafter, the process is returned to step S168. In step S168, a graph is displayed in accordance with the subject and period of the graph as designated.

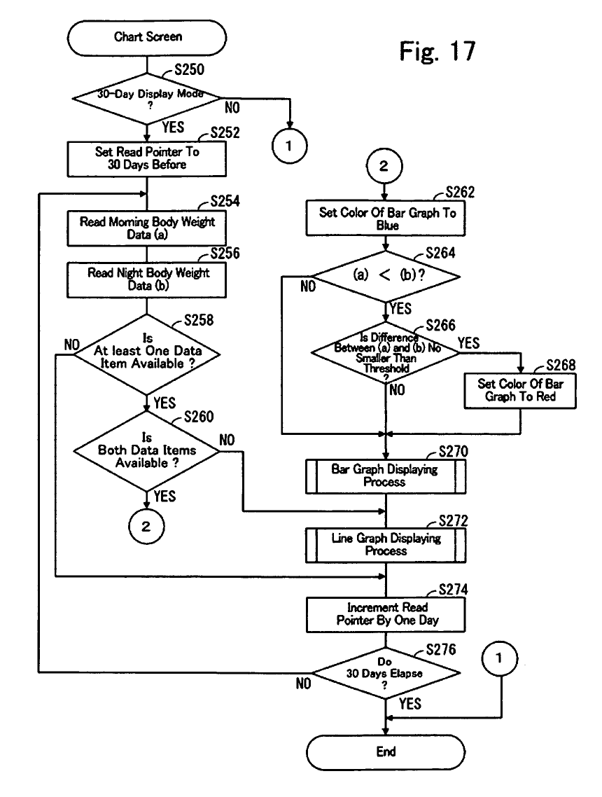

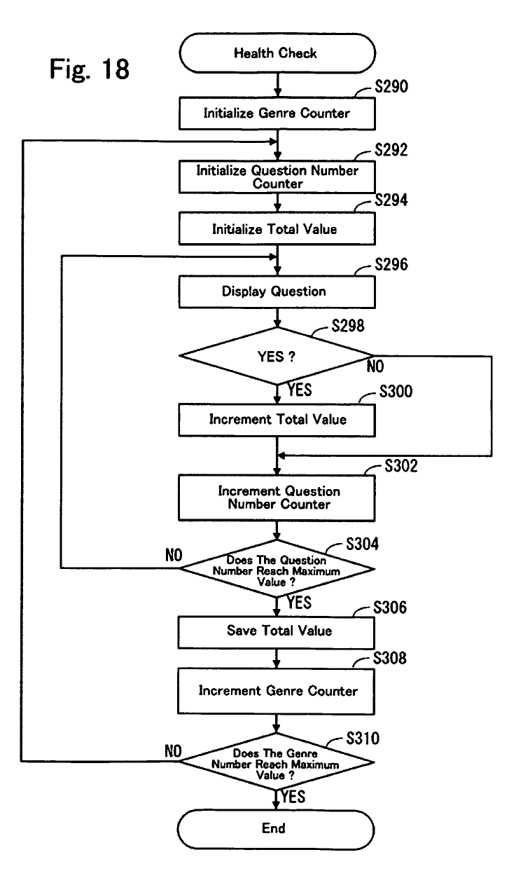

FIG. 17 is a flow chart for showing the details of the process of displaying the graph in step S168. At first, in step S250, it is determined whether or not the operation mode is the 30 day display mode. If it is the 30 day display mode, the process proceeds to step S252, otherwise the process ends.

The graph is displayed after step S252. More specifically speaking, the process proceeds as follows. In step S252, a read pointer is set 30 days before the current day. In step S254, the body weight data is read from the morning data of the day pointed to by the read pointer. In step S256, the body weight data of the night of the same day is read. It is determined in step S258 whether or not at least one data item of the body weight measured in the morning and night of the same day pointed to by the read pointer is available. If at least one is available, the process proceeds to step S260. Otherwise, i.e., if there is none of the data available, the process proceeds to step S274 without no processing.

It is determined in step S260 whether or not the body weight data items of both the morning and night of the same day pointed to by the read pointer is available. If the body weight data is available for both, the process proceeds to step S262. Otherwise, the process proceeds to step S272.

In step S262, the color of the vertical bar indicating the difference between the body weight in the night and the body weight in the morning is set to blue. Namely, the default color of the bar graph is set to blue.

In step S264, it is determined whether or not the body weight in the morning is smaller than the body weight in the night. If YES, the process proceeds to step S266. Otherwise, the process proceeds to step S270.

In step S266, it is determined whether or not the difference between the body weight in the night and the body weight in the morning is the threshold value or more. This threshold value is calculated with reference to the standard body weight X by a function that the threshold value=X×0.83%. If the difference is the threshold value or more, the process proceeds to step S268. Otherwise, the process proceeds to step S270.

In step S268, since the body weight in the night is greater than the body weight in the morning and the difference is the predetermined threshold value or more, it is judged that the increase in the body weight is not desirable and the color of the vertical bar is set to red. Thereafter, the process proceeds to step S270.

In step S270, the vertical bar is displayed in the vertical direction with the color as designated and the length corresponding to the difference between the body weight in the morning and the body weight in the night. The start point of the vertical bar is set to the position of the body weight in the morning in the ordinate, and the end point of the vertical bar is set to the position of the body weight in the night in the ordinate. In other words, the length of this vertical bar indicates the difference between the body weight in the morning and the body weight in the night.

In step S272, a line graph is displayed. The line graph is plotted by determining the point on the graph corresponding to each day pointed to by the read pointer such that, if both the body weights in the morning and night are available, the body weight in the morning is used, but if only one of these body weights is available, the available one is used. The pixels on the line segment between this point and the point of the previous day are plotted with a predetermined color. Thereafter, the process proceeds to step S274.

In step S274, the read pointer is incremented by one day. In the next step S276, it is determined whether or not the day pointed to by the read pointer is the day after 30 days has elapsed from the start of the plotting process as a result of incrementing the read pointer. If it is determined that 30 days has elapsed, the process of displaying the chart screen ends. If it is determined that 30 days has not elapsed yet, the process is returned to step S254 again, and the above process is repeated.

The chart display screen 380 of the body weight change shown in FIG. 10 is displayed by performing the above process for the period of 30 days.

FIG. 18 is a flow chart for showing the health check process to be performed in step S208 of FIG. 16. This process is started by manipulating “the enter key”+“the up and down keys” in the chart display screen. Incidentally, as has been discussed above, 30 questions are displayed to ask the user to successively answer these questions. These 30 questions are classified into six genres, and five questions are prepared for each genre.

In step S290, a genre counter for indicating the genre number of the question being asked is initialized.

In step S292, a question number counter for indicating the question number of the question being asked is initialized. Five questions are prepared for each genre and given question numbers 1 through 5 respectively.

In step S294, a total value indicating the number of preferred answers of the user's answers is initialized.

In step S296, of the questions belonging to the genre indicated by the genre counter, the question corresponding to the question number indicated by the question number counter is displayed. This screen is as illustrated in FIG. 11. When a question is displayed, the user selects the YES button 432 or the NO button 434 by pressing the arrow keys and the enter key as illustrated in FIG. 11. In the case of the present embodiment, all the questions are prepared such that it is preferred for the body weight management when the answer is “YES”.

In step S298, it is determined whether or not the user presses the enter key after selecting the YES button 432. If the user presses the enter key after selecting the YES button 432, the process proceeds to step S300. Otherwise, the process proceeds to step S302.

In step S300, the total value is incremented by one, and the process proceeds to step S302. In step S302, the question number counter is incremented by one, and the process proceeds to step S304. In step S304, it is determined whether or not the question number reaches the maximum value. More accurately speaking, it is determined whether or not the question number exceeds the number of the questions belonging to each genre. If it is determined that the question number is the maximum value, the process proceeds to step S306. Otherwise, the process is returned to step S296 in which the question corresponding to the next question number is displayed, and the above process is repeated.

If it is determined in step S304 that the question number reaches the maximum value, the total value is saved in step S306. The score of the user is determined by this value with respect to the genre indicated by the value of the genre counter. After step S306, the process proceeds to step S308.

In step S308, the genre counter is incremented. It is determined in step S310 whether or not the value of the genre counter exceeds the maximum value as a result of the increment. In other words, it is determined whether or not the value of the genre counter exceeds 6. If it is determined that the value of the genre counter exceeds 6, the health check process ends. Otherwise, the process is returned to step S292, and the above process is repeated from step S292.

In step S210 shown in FIG. 16, the radar chart shown in FIG. 12 is displayed on the basis of the score (total value) of the user which is obtained for each genre as described above. At this time, the previous result is also displayed together in the form of the radar chart.

FIG. 19 is a flow chart for showing the process of displaying the course and achievement in step S160 of FIG. 15. As shown in FIG. 19, in step S330, it is determined whether or not the operation mode is the diet mode. The course and achievement is displayed only in the diet mode. Accordingly, if the result of determination is NO in step S330, this process ends. Conversely, if the result of determination is YES in step S330, the process proceeds to step S332.

In step S332, it is determined whether or not two months have just elapsed after starting diet. If two months have just elapsed, the process proceeds to step S350, otherwise proceeds to step S334. If the body weight has not decreased by 1 kg or more yet two months after starting diet, the diet is likely to fail and end after six months. It is therefore determined in step S350 whether or not the body weight has decreased by 1 kg or more relative to the body weight just before diet. If it is determined that the body weight has decreased by 1 kg or more, a diet encouraging indication is displayed in step S352 to indicate that the diet is likely to succeed. Conversely, if it is determined that the body weight has decreased by less than 1 kg or increased, a diet failure indication is displayed to indicate that the diet is likely to fail. An example of this display is illustrated in FIG. 13. After steps S352 and S354, the process proceeds to step S342. The process after step S342 will be described below.

On the other hand, in step S332, it is determined that two months have not just elapsed after starting diet, the process proceeds to step S334. In step S334, it is determined whether or not six months has elapsed after starting diet. If the result of determination is YES, the process proceeds to step S336, otherwise this process ends.

In step S336, it is determined whether or not the current body weight exceeds the target body weight. If the current body weight exceeds the target body weight, the process proceeds to step S340, otherwise proceeds to step S338.

In step S340, the diet failure indication is displayed, the process proceeds to step S342. On the other hand, in step S338, the diet succeed indication is displayed, the process proceeds to step S342.

In step S342, the system waits for some key input operation. If there are some key input operation, this process ends.

Meanwhile, in the case of the present embodiment, the decreasing amount of the body weight and the predetermined fixed threshold value (1 kg) are compared in order to make a determination 60 days after starting diet, i.e., a prediction of whether or not the diet is likely to succeed. However, the present invention is not limited to such a specific embodiment. For example, the threshold value may be determined in accordance with a function of the gender and body weight.

FIG. 20 to FIG. 23 show a flow chart of the process of displaying the body weight tendency to be performed in steps S162 and S170 of FIG. 15. As shown in FIG. 20, the average value from 29 days before to the current day is calculated in step S360 by the process of displaying the body weight tendency. This value is referred here to as X30. In step S362, the average value of the body weight from 59 days before to 30 days before is calculated. This value is referred here to as Y30. In step S364, it is determined whether or not the body weight measured 29 days before exceeds 60 kg. If it exceeds 60 kg, the process proceeds to step S368. Otherwise, the process proceeds to step S366.

In step S366, a reference increment is read from the table 4 in correspondence with the body weight of less than or equal to 60 kg and the number of days which is equal to 30. After step S366, the process proceeds to step S370. On the other hand, in step S368, a reference increment is read from the table 4 in correspondence with the body weight of greater than 60 kg and the number of days which is equal to 30. After step S368, the process proceeds to step S370.

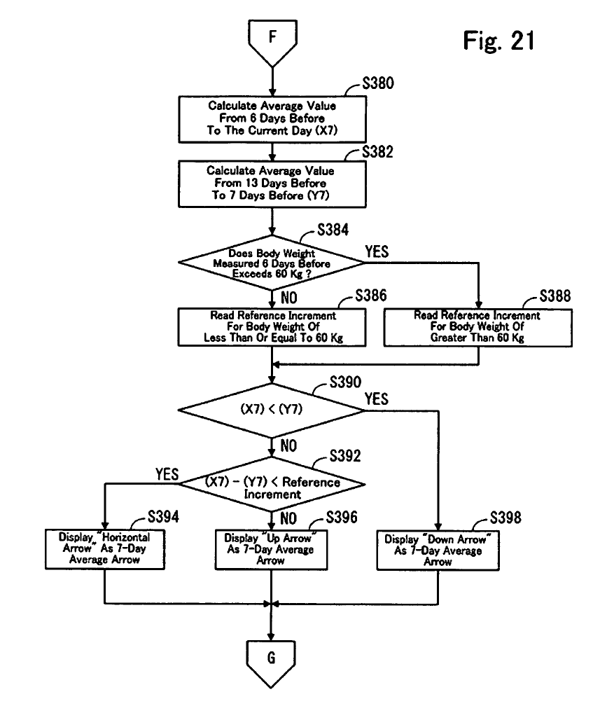

In step S370, it is determined whether or not the value X30 calculated in step S360 and the value Y30 calculated in step S362 satisfy the relation that X30<Y30. If these values satisfy this relation, the process proceeds to step S378. Otherwise, the process proceeds to step S372. In step S378, it is determined to display “a down arrow”, as a 30-day average arrow, which indicates that the body weight tends to decrease, and the process proceeds to step S380 of FIG. 21.

In step S372, by comparing X30-Y30 and the reference increment which is read from the table in step S366 or S368, it is determined whether or not the relation that X30-Y30<the reference increment is satisfied. If satisfied, the process proceeds to step S374, otherwise proceeds to step S376. In step S374, it is determined to display “a horizontal arrow”, as a 30-day average arrow, and the process proceeds to step S380 of FIG. 21. In step S376, it is determined to display “an up arrow” pointing to the upper right, as a 30-day average arrow, and the process proceeds to step S380 of FIG. 21. An arrow indicating the change tendency of the 30-day average body weight is determined by the above process.

Next, the arrow indicating the change tendency of the 7-day average body weight is determined by the following process. As shown in FIG. 21, the average value of the body weight from 6 days before to the current day is calculated in step S380. This value is referred here to as X7. In step S382, the average value of the body weight from 13 days before to 7 days before is calculated. This value is referred here to as Y7. In step S384, it is determined whether or not the body weight measured 6 days before exceeds 60 kg. If it exceeds 60 kg, the process proceeds to step S388. Otherwise, the process proceeds to step S386.

In step S386, a reference increment is read from the table 4 in correspondence with the body weight of less than or equal to 60 kg and the number of days which is equal to 7. After step S386, the process proceeds to step S390. On the other hand, in step S388, a reference increment is read from the table 4 in correspondence with the body weight of greater than 60 kg and the number of days which is equal to 7. After step S388, the process proceeds to S390.

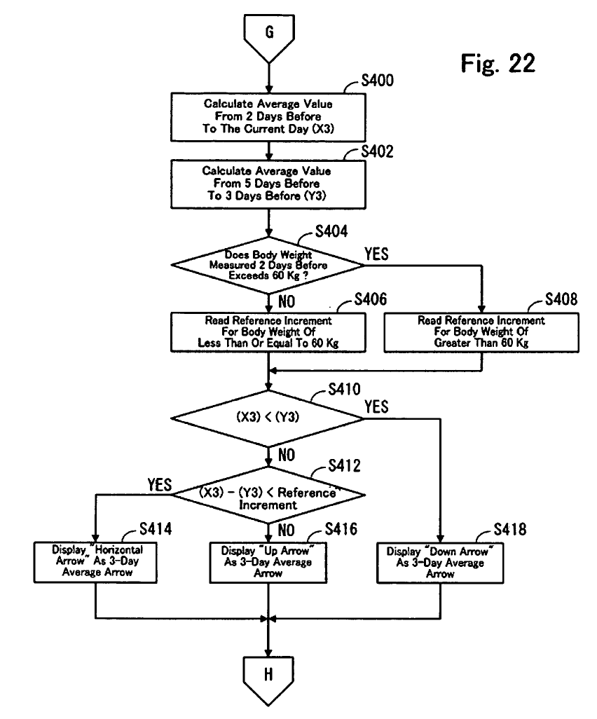

In step S390, it is determined whether or not the value X7 calculated in step S380 and the value Y7 calculated in step S382 satisfy the relation that X7<Y7. If these values satisfy this relation, the process proceeds to step S398. Otherwise, the process proceeds to step S392. In step S398, it is determined to display “a down arrow”, as a 7-day average arrow, which indicates that the body weight tends to decrease, and the process proceeds to step S400 of FIG. 22.

In step S392, by comparing X7−Y7 and the reference increment which is read from the table in step S386 or S388, it is determined whether or not the relation that X7−Y7<the reference increment is satisfied. If satisfied, the process proceeds to step S394, otherwise proceeds to step S396. In step S394, it is determined to display “a horizontal arrow”, as a 7-day average arrow, and the process proceeds to step S400 of FIG. 22. In step S396, it is determined to display “an up arrow” pointing to the upper right, as a 7-day average arrow, and the process proceeds to step S400 of FIG. 22. An arrow indicating the change tendency of the 7-day average body weight is determined by the above process.

Next, the arrow indicating the change tendency of the 3-day average body weight is determined by the following process. As shown in FIG. 22, the average value of the body weight from 2 days before to the current day is calculated in step S400. This value is referred here to as X3. In step S402, the average value of the body weight from 5 days before to 3 days before is calculated. This value is referred here to as Y3. In step S404, it is determined whether or not the body weight measured 2 days before exceeds 60 kg. If it exceeds 60 kg, the process proceeds to step S408. Otherwise, the process proceeds to step S406.

In step S406, a reference increment is read from the table 4 in correspondence with the body weight of less than or equal to 60 kg and the number of days which is equal to 3. After step S406, the process proceeds to step S410. On the other hand, in step S408, a reference increment is read from the table 4 in correspondence with the body weight of greater than 60 kg and the number of days which is equal to 3. After step S408, the process proceeds to S410.

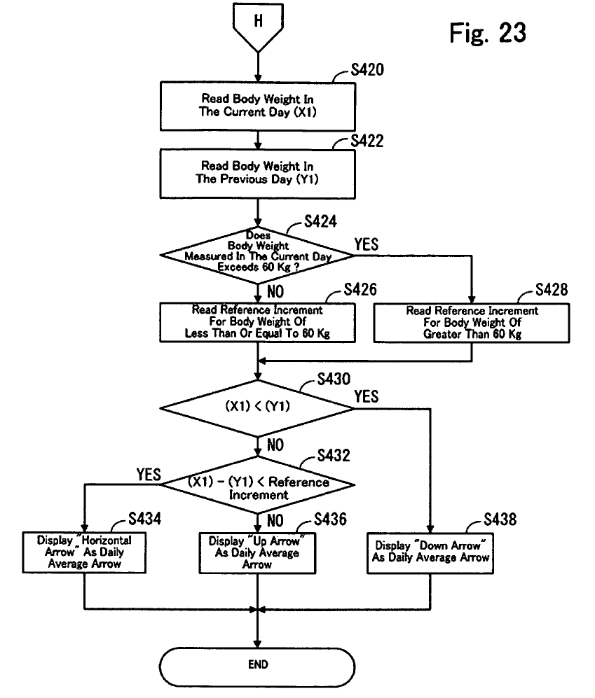

In step S410, it is determined whether or not the value X3 calculated in step S400 and the value Y3 calculated in step S402 satisfy the relation that X3<Y3. If these values satisfy this relation, the process proceeds to step S418. Otherwise, the process proceeds to step S412. In step S418, it is determined to display “a down arrow”, as a 3-day average arrow, which indicates that the body weight tends to decrease, and the process proceeds to step S420 of FIG. 23. In step S412, by comparing X3−Y3 and the reference increment which is read from the table in step S406 or S408, it is determined whether or not the relation that X3−Y3<the reference increment is satisfied. If satisfied, the process proceeds to step S414, otherwise proceeds to step S416. In step S414, it is determined to display “a horizontal arrow”, as a 3-day average arrow, and the process proceeds to step S420 of FIG. 23. In step S416, it is determined to display “an up arrow” pointing to the upper right, as a 3-day average arrow, and the process proceeds to step S420 of FIG. 23. An arrow indicating the change tendency of the 3-day average body weight is determined by the above process.

Next, the arrow indicating the change tendency of the body weight in the current day is determined by the following process. As shown in FIG. 23, the body weight of the current day is read in step S420. This value is referred here to as X1. In step S422, the body weight of the previous day is read. This value is referred here to as Y1. In step S424, it is determined whether or not the body weight of the current day exceeds 60 kg. If it exceeds 60 kg, the process proceeds to step S428. Otherwise, the process proceeds to step S426.

In step S426, a reference increment is read from the table 4 in correspondence with the body weight of less than or equal to 60 kg and the number of days which is equal to 1. After step S426, the process proceeds to step S430. On the other hand, in step S428, a reference increment is read from the table 4 in correspondence with the body weight of greater than 60 kg and the number of days which is equal to 1. After step S428, the process proceeds to step S430.

In step S430, it is determined whether or not the value X1 obtained in step S420 and the value Y1 obtained in step S422 satisfy the relation that X1<Y1. If these values satisfy this relation, the process proceeds to step S438. Otherwise, the process proceeds to step S432. In step S438, it is determined to display “a down arrow”, as a current day arrow, which indicates that the body weight tends to decrease, and the body weight tendency displaying process ends.

In step S432, by comparing X1−Y1 and the reference increment which is read from the table in step S426 or S428, it is determined whether or not the relation that X1−Y1<the reference increment is satisfied. If satisfied, the process proceeds to step S434, otherwise proceeds to step S436. In step S434, it is determined to display “a horizontal arrow”, as a current day arrow, and the body weight tendency displaying process ends. In step S436, it is determined to display “an up arrow” pointing to the upper right, as a current day arrow, and the body weight tendency displaying process ends. An arrow indicating the change tendency of the body weight in the current day is determined by the above process.

In this way, the body weight tendency screen is displayed in steps S162 and S170 of FIG. 15. Incidentally, in the case of the chart screen processed in step S168 of FIG. 15 and so forth, the arrow indication determined in this way is displayed as a reduced image in a corner of the screen (refer to FIG. 10).

By the process as described above, it is possible to display the change tendency of body weight to the user in the long, medium and short term together with the current change tendency with arrows in a form which can be more easily and intuitively understood. Understanding is further facilitated by drawing these arrows with colors associated with the change tendency of body weight. Also, the indication by arrows is easily understandable even if the arrows are reduced in size, and thereby the size-reduced image of the body weight tendency indication can be used in a variety of screens.

Meanwhile, the present invention is not limited to the above embodiment, and a variety of variations and modifications may be effected without departing from the spirit and scope thereof, as described in the following exemplary modifications.

In the case of the present embodiment, as illustrated in FIG. 10, the line graph 390 is plotted by the use of the body weights in the morning. However, the present invention is not limited to this specific embodiment, but the body weights in the night can be used instead for plotting the line graph 390. In this case, while the method of displaying vertical bars may be performed in the same manner as illustrated in FIG. 10, the directions of the vertical bars may be reversed (i.e., a vertical bar is displayed to extend in the downward direction if the body weight in the night is greater than the body weight in the morning).

Meanwhile, in the case of the example shown in FIG. 10, the vertical bars indicative of the difference between the body weights in the morning and night are drawn on the line graph which is plotted by the use of the body weights in the morning. However, the present invention is not limited to this particular embodiment, but any displays can be used, as long as the difference between the body weights in the morning and night is indicated with the line graph as a reference graph the line graph as a reference. An alternative can be provided by drawing a line graph plotted with the body weights in the morning and a line graph plotted with the body weights in the night, and daily changing the color of each area between these line graphs in accordance with the difference between the body weight in the night and the body weight in the morning in the same manner as in the above described embodiment. Also, the color of the line graph plotted with the body weights in the night may be daily changed in accordance with the differential body weight.

While a cartridge type is employed in the above cases, it is also possible to implement the respective functions of the cartridge 3 within the adapter 1 and dispense with the cartridge 3.

While the embodiments disclosed herein are provided only for illustrative purposes, the present invention is not limited to the embodiment as described above. The scope of the present invention is defined by the respective patent claims taking into consideration the detailed description of the invention, and includes any type of modifications within the scope in view of the description therein under the doctrine of equivalents.

### INDUSTRIAL APPLICABILITY

The present invention can be used in the industry manufacturing the health care support system for managing body weight, body fat percentage, blood pressure, blood glucose level, number of pulses, body temperature, dimensions of predetermined areas of body, number of steps, consumed calories, taken calories and the like, and the service industry making use of such a system.

1. A system for supporting health management comprising:
a storing unit operable to store a measurement value of predetermined biometric information in association with the measurement date thereof; and
a symbol assignment unit operable to calculate the change tendencies of the measurement values of the biometric information stored in said storing unit over a plurality of periods each of which consists of previous days including a predetermined measurement day as the last day of the each period, and assign one symbol of a plurality of types of symbols, which are prepared in advance, to each of the plurality of periods in accordance with the change tendency as calculated;
a display unit operable to display the symbols assigned to the plurality of periods by said symbol assignment unit respectively in the same display screen.

2. The system for supporting health management as claimed in claim 1 wherein the symbols include arrow symbols having inclinations which reflect the change tendencies of the measurement value of the biometric information.

3. The system for supporting health management as claimed in claim 1 wherein the symbols are given different colors respectively in accordance with the calculated change tendencies.

4. The system for supporting health management as claimed in claim 1 wherein the predetermined biometric information is body weight, body fat percentage, blood pressure, blood glucose level, a dimension of a predetermined area of body, number of pulses, body temperature, number of steps, consumed calories, taken calories, or any combination thereof.

5. The system for supporting health management as claimed in claim 1 wherein said symbol assignment unit comprises:
a change tendency calculating unit operable to calculate the change tendencies of the measurement values of the biometric information stored in said storing unit over the plurality of periods each of which consists of the previous days including the predetermined measurement day as the last day of the each period; and
an assignment unit operable to assign one symbol of the plurality of types of symbols, which are prepared in advance, to each of the plurality of periods in accordance with the change tendency calculated by said change tendency calculating unit, and wherein
said change tendency calculating unit comprises:
a first moving average calculating unit operable to calculate the moving average of the biometric information, for each of the plurality of periods, over a period whose length is same as that of the each of the plurality of periods and whose last day is one day before the first day of the each of the plurality of periods;
a second moving average calculating unit operable to calculate the moving average of the biometric information, for each of the plurality of periods, over a period whose length is same as that of the each of the plurality of periods and whose last day is the predetermined measurement day; and
a calculating unit operable to calculate, for each of the plurality of periods, the difference between the moving averages calculated by said second moving average calculating unit and said first moving average calculating unit.

6. The system for supporting health management as claimed in claim 5 wherein said assignment unit includes
a unit operable to assign, for each of the plurality of periods, one of the plurality of types of symbols to the each of the plurality of periods in accordance with which of a plurality of predetermined ranges includes the difference between the moving averages of the each of the plurality of periods calculated by said unit operable to calculate the difference.

7. The system for supporting health management as claimed in claim 6 wherein the plurality of predetermined ranges is given as a function of the biometric information stored in said storing unit and the length of the period to be considered.

8. A system for supporting health management comprising:
a storing unit operable to daily store a measurement value of predetermined biometric information in a first time slot and a measurement value of the predetermined biometric information in a second time slot different from the first time slot respectively in association with the measurement date thereof;
a first graph displaying unit operable to display a graph in a first graph form showing the change in the measurement value in the first time slot stored in said storage unit for a predetermined period; and
a second graph displaying unit operable to graph the difference between the measurement value of the predetermined biometric information in the first time slot and the measurement value of the predetermined biometric information in the second time slot stored in said storage unit in the each measurement date within the predetermined period in a second graph form, and display the graph of the difference to be superimposed over the graph displayed by said first graph displaying unit,
wherein said second graph displaying unit displays a graph, using the position of the measurement value in the first time slot for each measurement date as a base point, to show the difference between the measured value in the second time slot and the measured value in the first time slot in the each measurement date.

9. The system for supporting health management as claimed in claim 8 wherein said second graph displaying unit comprises:
a determination unit operable to determine whether or not the difference in each measurement date satisfies a predetermined condition; and
an overlapping displaying unit operable to graph the difference in the each measurement date within the predetermined period in the second graph form, and display the graph of the difference to be superimposed over the graph displayed by said first graph displaying unit,
wherein said overlapping displaying unit displays the graph using the position of the measurement value in the first time slot for each measurement date as a base point and using a different color in accordance with the result of determination by said determination unit.

10. The system for supporting health management as claimed in claim 9 wherein the predetermined biometric information is body weight, body fat percentage, blood pressure, blood glucose level, a dimension of a predetermined area of body, number of pulses, body temperature, number of steps, consumed calories, taken calories, or any combination thereof,
wherein the first time slot is selected as a time slot which is earlier than the second time slot in each measurement date, and
wherein said determination unit includes a unit operable to determine, for each measurement date, whether or not the difference of the each measurement date is greater than a predetermined threshold value.

11. The system for supporting health management as claimed in claim 10 wherein said overlapping displaying unit uses a color of red for graphing the difference in response to the determination that the difference of the measurement date is greater than the predetermined threshold value.

12. The system for supporting health management as claimed in claim 10 further comprising a unit operable to display a particular screen, which is prepared in advance for managing body weight, in response to the determination by said determination unit that the difference of a certain measurement date is greater than the predetermined threshold value.

13. The system for supporting health management as claimed in claim 10 further comprising a unit operable to calculate the predetermined threshold value as a function of the biometric information of a user stored in said storage unit.

14. The system for supporting health management as claimed in claim 10 wherein said overlapping displaying unit uses a color of red for graphing the difference in response to the determination by said determination unit that the difference of the measurement date is greater than the predetermined threshold value.

15. The system for supporting health management as claimed in claim 8 wherein the first graph form is a line graph connecting daily measurement values.

16. The system for supporting health management as claimed in claim 8 wherein the second graph form is a vertical bar graph consisting of bars each of which has a vertical length in proportion to the daily difference.

17. A recording medium storing a computer program for causing, when executed by a computer connected to a display device, the computer to function as a system for supporting health management, said system comprising:
a storing unit operable to store measurement values of predetermined biometric information measured at predetermined intervals in association with the measurement date thereof;
a symbol assignment unit operable to calculate the change tendencies of the measurement values of the biometric information stored in said storing unit over a plurality of periods each of which consists of previous days including a predetermined measurement day as the last day of the each period, and assign one symbol of a plurality of types of symbols, which are prepared in advance, to each of the plurality of periods in accordance with the change tendency as calculated; and
a display signal generation unit operable to generate a signal for displaying the symbols assigned to the plurality of periods by said symbol assignment unit respectively in the same display screen of the display device.

18. The recording medium as claimed in claim 17 wherein the symbols include arrow symbols having inclinations which reflect the change tendencies of the measurement value of the biometric information.

19. The recording medium as claimed in claim 17 wherein the symbols are given different colors respectively in accordance with the calculated change tendencies.

20. The recording medium as claimed in claim 17 wherein the predetermined biometric information is body weight, body fat percentage, blood pressure, blood glucose level, a dimension of a predetermined area of body, number of pulses, body temperature, number of steps, consumed calories, taken calories, or any combination thereof.

21. The recording medium as claimed in claim 17 wherein said symbol assignment unit comprises:
a change tendency calculating unit operable to calculate the change tendencies of the measurement values of the biometric information stored in said storing unit over the plurality of periods each of which consists of the previous days including the predetermined measurement day as the last day of the each period; and
an assignment unit operable to assign one symbol of the plurality of types of symbols, which are prepared in advance, to each of the plurality of periods in accordance with the change tendency calculated by said change tendency calculating unit, and wherein said change tendency calculating unit comprises:
a first moving average calculating unit operable to calculate the moving average of the biometric information, for each of the plurality of periods, over a period whose length is same as that of the each of the plurality of periods and whose last day is one day before the first day of the each of the plurality of periods;
a second moving average calculating unit operable to calculate the moving average of the biometric information, for each of the plurality of periods, over a period whose length is same as that of the each of the plurality of periods and whose last day is the predetermined measurement day; and
a calculating unit operable to calculate, for each of the plurality of periods, the difference between the moving averages calculated by said second moving average calculating unit and said first moving average calculating unit.

22. The recording medium as claimed in claim 21 wherein said assignment unit includes a unit operable to assign, for each of the plurality of periods, one of the plurality of types of symbols to the each of the plurality of periods in accordance with which of a plurality of predetermined ranges includes the difference between the moving averages of the each of the plurality of periods calculated by said unit operable to calculate the difference.

23. The recording medium as claimed in claim 22 wherein the plurality of predetermined ranges is given as a function of the biometric information stored in said storing unit and the length of the period to be considered.

24. A recording medium storing a computer program for causing, when executed by a computer connectable to a display device, the computer to function as a system for supporting health management, said system comprising:
a storing unit operable to daily store a measurement value of predetermined biometric information in a first time slot a measurement value of the predetermined biometric information in a second time slot different from the first time slot respectively in association with the measurement dates thereof;
a first graph displaying unit operable to display a graph in a first graph form showing the change in the measurement value in the first time slot stored in said storage unit for a predetermined period; and
a second graph displaying unit operable to graph the difference between the measurement value of the predetermined biometric information in the first time slot and the measurement value of the predetermined biometric information in the second time slot stored in said storage unit in the each measurement date within the predetermined period in a second graph form, and display the graph of the difference to be superimposed over the graph displayed by said first graph displaying unit,
wherein said second graph displaying unit displays a graph, using the position of the measurement value in the first time slot for each measurement date as a base point, to show the difference between the measured value in the second time slot and the measured value in the first time slot in the each measurement date.

25. The recording medium as claimed in claim 24 wherein said second graph displaying unit comprises:
a determination unit operable to determine whether or not the difference in each measurement date satisfies a predetermined condition; and
an overlapping displaying unit operable to graph the difference in the each measurement date within the predetermined period in the second graph form, and display the graph of the difference to be superimposed over the graph displayed by said first graph displaying unit,
wherein said overlapping displaying unit displays the graph using the position of the measurement value in the first time slot for each measurement date as a base point and using a different color in accordance with the result of determination by said determination unit.

26. The recording medium as claimed in claim 25 wherein the predetermined biometric information is body weight, body fat percentage, blood pressure, blood glucose level, a dimension of a predetermined area of body, number of pulses, body temperature, number of steps, consumed calories, taken calories, or any combination thereof,
wherein the first time slot is selected as a time slot which is earlier than the second time slot in each measurement date, and
wherein said determination unit includes a unit operable to determine, for each measurement date, whether or not the difference of the each measurement date is greater than a predetermined threshold value.

27. The recording medium as claimed in claim 26 wherein said overlapping displaying unit uses a color of red for graphing the difference in response to the determination that the difference of the measurement date is greater than the predetermined threshold value.

28. The recording medium as claimed in claim 26 wherein said system further comprises a unit operable to display a comment screen, which is prepared in advance for managing body weight, in response to the determination by said determination unit that the difference of a certain measurement date is greater than the predetermined threshold value.

29. The recording medium as claimed in claim 26 wherein said system further comprises a unit operable to calculate the predetermined threshold value as a function of the biometric information of a user stored in said storage unit.

30. The recording medium as claimed in claim 26 wherein said overlapping displaying unit uses a color of red for graphing the difference in response to the determination by said determination unit that the difference of the measurement date is greater than the predetermined threshold value.

31. The recording medium as claimed in claim 24 wherein the first graph form is a line graph connecting daily measurement values.

32. The recording medium as claimed in claim 24 wherein the second graph form is a vertical bar graph consisting of bars each of which has a vertical length in proportion to a daily difference.

33. A system for supporting health management comprising:
a measuring unit operable to measure predetermined biometric information;
a storing unit operable to store a measurement value of the predetermined biometric information in association with the measurement date thereof;
a determining unit operable to determine whether or not there is an enquiry from an external information processing unit; and
a transmitting unit operable to transmit the measurement value, which is stored in the storing unit and associated with the measurement date, to the external information processing unit when an enquiry is received from the external information processing unit.

34. The system for supporting health management as claimed in claim 33 wherein the determining unit determines, at predetermined intervals, whether or not there is an enquiry from the external information processing unit.

35. The system for supporting health management as claimed in claim 33 wherein the transmitting unit transmits the measurement value, which is stored in the storing unit and associated with the measurement date, by wireless communication.

36. The system for supporting health management as claimed in claim 33 wherein the storing unit stores the measurement value in association with each of a plurality of users, and
wherein the transmitting unit transmits the measurement value associated with the measurement date to the external information processing unit together with information identifying the user corresponding to the measurement.

37. The system for supporting health management as claimed in claim 33 wherein the predetermined biometric information is body weight, body fat percentage, blood pressure, blood glucose level, number of pulses, body temperature, number of steps, consumed calories, taken calories, or any combination thereof.

38. A system for supporting health management comprising:
a transmitting unit operable to transmit an enquiry to a biometric information measuring unit which is externally located and serves to measure predetermined biometric information;
a receiving unit operable to receive a measurement value of the predetermined biometric information which is associated with the measurement date thereof and transmitted from the biometric information measuring unit in response to the enquiry; and
a storing unit operable to store the measurement value as received in association with the measurement date thereof.

39. The system for supporting health management as claimed in claim 38 wherein the transmitting unit transmits the enquiry to the biometric information measuring unit in response to a power-up operation.

40. The system for supporting health management as claimed in claim 38 wherein the transmitting unit transmits the enquiry to the biometric information measuring unit at predetermined intervals.

41. The system for supporting health management as claimed in claim 38 wherein the transmitting unit transmits the enquiry by wireless communication, and wherein the receiving unit receives the measurement value associated with the measurement date by wireless communication.

42. The system for supporting health management as claimed in claim 38 further comprising a video signal generating unit operable to generate a video signal on the basis of the measurement value as received and associated with the measurement date thereof.

43. The system for supporting health management as claimed in claim 42 wherein the video signal generating unit generates, for each user, the video signal based on the received measurement value associated with the measurement date in accordance with information identifying the user which is received.

44. The system for supporting health management as claimed in claim 38 wherein the predetermined biometric information is body weight, body fat percentage, blood pressure, blood glucose level, number of pulses, body temperature, number of steps, consumed calories, taken calories, or any combination thereof.

45. A system for supporting health management comprising:
a biometric information measuring unit operable to measure predetermined biometric information; and
an information processing unit which is provided external to the biometric information measuring unit and operable to receive the predetermined biometric information from the biometric information measuring unit and perform processes in accordance with a computer program,
the biometric information measuring unit comprising:
a measuring unit operable to measure the predetermined biometric information;
a first storing unit operable to store a measurement value of the predetermined biometric information in association with the measurement date thereof;
a determining unit operable to determine whether or not there is an enquiry from the external biometric information measuring unit; and
a first transmitting unit operable to transmit the measurement value, which is stored in the first storing unit and associated with the measurement date, to the information processing unit when the enquiry is received from the information processing unit which is externally provided,
the information processing unit comprising:
a second transmitting unit operable to transmit the enquiry to the biometric information measuring unit;
a receiving unit operable to receive the measurement value which is associated with the measurement date thereof and transmitted from the biometric information measuring unit in response to the enquiry; and
a second storing unit operable to store the measurement value in association with the measurement date thereof which is received.

46. A recording medium storing a computer program for causing, when executed by a computer connected to a display device, the computer to function as a system for supporting health management, the system comprising:
a measuring unit operable to measure predetermined biometric information;
a storing unit operable to store a measurement value of the predetermined biometric information in association with the measurement date thereof;
a determining unit operable to determine whether or not there is an enquiry from an external information processing unit; and
a transmitting unit operable to transmit the measurement value, which is stored in the storing unit and associated with the measurement date, to the external information processing unit when the enquiry is received from the external information processing unit.

47. A recording medium storing a computer program for causing, when executed by a computer connected to a display device, the computer to function as a system for supporting health management, the system comprising:
a transmitting unit operable to transmit an enquiry to a biometric information measuring unit which is externally located and serves to measure predetermined biometric information;
a receiving unit operable to receive a measurement value of the predetermined biometric information which is associated with the measurement date thereof and transmitted from the biometric information measuring unit in response to the enquiry; and
a storing unit operable to store the measurement value in association with the measurement date thereof which is received.
